(19) United States
(12) Patent Application Publication
(10) Pub. No.: US 2025/0248770 A1
(43) Pub. Date: Aug. 7, 2025

(54) SYSTEM AND METHOD FOR ILLUSTRATING A POSE OF AN OBJECT

(71) Applicant: Medtronic, Inc., Minneapolis, MN (US)

(72) Inventors: Elliot C. Schmidt, Minneapolis, MN (US); Can Cinbis, Salt Lake City, UT (US); Kenneth C. Gardeski, Inverness, FL (US); Joshua J. Blauer, White Bear Township, MN (US); Varun A. Bhatia, Minneapolis, MN (US); Ryan P. Lahm, Lino Lakes, MN (US); Brett D. Jackson, Shoreview, MN (US); Tarek D. Haddad, Minneapolis, MN (US)

(21) Appl. No.: 19/191,751

(22) Filed: Apr. 28, 2025

Related U.S. Application Data
(63) Continuation-in-part of application No. PCT/IB2023/060058, filed on Oct. 6, 2023.
(63) Continuation-in-part of application No. PCT/IB2023/060059, filed on Oct. 6, 2023.
(63) Continuation-in-part of application No. PCT/IB2023/060060, filed on Oct. 6, 2023.
(60) Provisional application No. 63/420,274, filed on Oct. 28, 2022.
(60) Provisional application No. 63/420,315, filed on Oct. 28, 2022.
(60) Provisional application No. 63/420,328, filed on Oct. 28, 2022.

(51) Int. Cl. A61B 34/20; A61B 34/10; A61B 90/00; G06T 7/20; G06T 7/70

(52) U.S. Cl. CPC A61B 34/20; A61B 34/10; A61B 90/37; G06T 7/20; G06T 7/70; A61B 2034/102; A61B 2034/105

(57) ABSTRACT

Disclosed is a system to display a representation of an instrument. The representation of the instrument may be displayed relative to a representation of a patient. The representation of the patient may be fit in real-time to represent positions of two or portions of the patient relative to one another.

This application is a continuation-in-part of International Patent Application No. PCT/IB2023/060058, filed Oct. 6, 2023, which claims the benefit of U.S. Provisional Patent Application No. 63/420,274, filed Oct. 28, 2022. This application is also a continuation-in-part of International Patent Application No. PCT/IB2023/060059, filed Oct. 6, 2023, which claims the benefit of U.S. Provisional Patent Application No. 63/420,315, filed Oct. 28, 2022. This application is also a continuation-in-part of International Patent Application No. PCT/IB2023/060060, filed Oct. 6, 2023, which claims the benefit of U.S. Provisional Patent Application No. 63/420,328, filed Oct. 28, 2022. Each of the aforementioned applications is incorporated herein by reference in its entirety.

### FIELD

The present disclosure relates to acquisition of image data of a subject, and particularly to acquisition and display of image data collected from a tracked imaging system.

### BACKGROUND

This section provides background information related to the present disclosure which is not necessarily prior art.

An imaging system can be used to image various portions of a subject. The subject can include a patient, such as a human patient. The portions selected to be imaged can be internal portions that are covered by skin or other tissue. A location of imaged portions of the subject may be selected to be known within the imaging system. The locations can be defined or established relative to instruments placed in the subject (e.g., a location of a heart wall relative to a catheter) or a location of the imaged portion relative to the instrument acquiring the image data.

An imaging modality may be used to generate image data that are used to render images of an interior of the subject. For example, x-rays may be used to generate image data of an interior of a subject. The images may be displayed for viewing by a user.

### SUMMARY

This section provides a general summary of the disclosure, and is not a comprehensive disclosure of its full scope or all of its features.

The subject disclosure relates to illustrating a position, including a pose of an instrument relative to a patient. The patient may be a human or nonhuman patient. Further the patient or subject may be a non-living object where the location of an instrument within the subject is selected to be tracked.

A representation of the patient may be fit or morphed to illustrate or represent a size of the patient. Therefore, relative portions of the patient, such as relative portions of the anatomy, are determined or known relative to one another based upon the patient that is the current patient during a procedure. An avatar or indicia of the patient may be generated or altered to a specific patient substantially in real time during a procedure.

An instrument can be tracked relative to the patient. A representation of the instrument may be superimposed on the avatar of the patient. The avatar of the patient may provide a visual illustration of various portions of the anatomy of the patient without requiring image data being acquired of the patient. The display being viewed by the user may be generated without requiring image data of the patient, such as fluoroscopy image data.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, not all possible implementations, and are not intended to limit the scope of the present disclosure.

FIG. 1 is an environmental view of a patient with an imaging and navigation system;

FIG. 2 is a screen shot of a representation of an instrument at a tracked pose relative to an avatar of the patient, according to various embodiments;

FIG. 3 is a schematic view of a patient in an operating theater;

FIG. 4 is a schematic view of a user and a patient in an operating theater, according to various embodiments;

FIG. 5 is a detailed view of an instrument being moved relative to a patient;

FIG. 6 is a screen shot of a display illustrating a position of an instrument relative to an avatar;

FIG. 7 is a flowchart of a method, according to various embodiments;

FIG. 8 is a schematic illustration of an atlas and an image;

FIG. 9 is an illustration of images in two planes of a heart, according to various embodiments including ultrasound;

FIGS. 10A and 10B illustrate movement of a tracked instrument over time;

FIG. 11 is a flowchart illustrating a method of showing movement of a tracked instrument over time, according to various embodiments;

FIG. 12A illustrates a tracked pose of an instrument relative to an image in a plane in two orientations, according to various embodiments;

FIG. 12B illustrates a tracked pose of an instrument relative to an image in a plane in two orientations, according to various embodiments;

FIG. 13 is a schematic illustration of a magnet;

FIG. 14 is a schematic illustration of the magnet relative to various instruments, according to various embodiments;

FIG. 15 is a schematic illustration of a magnetic sensing assembly;

FIG. 16 is a schematic illustration of an operating room with magnetic sensing assemblies, according to various embodiments; and

FIG. 17 is a flowchart illustrating a method of tracking an instrument with a permanent magnetic field.

Corresponding reference numerals indicate corresponding parts throughout the several views of the drawings.

### DETAILED DESCRIPTION

Example embodiments will now be described more fully with reference to the accompanying drawings.

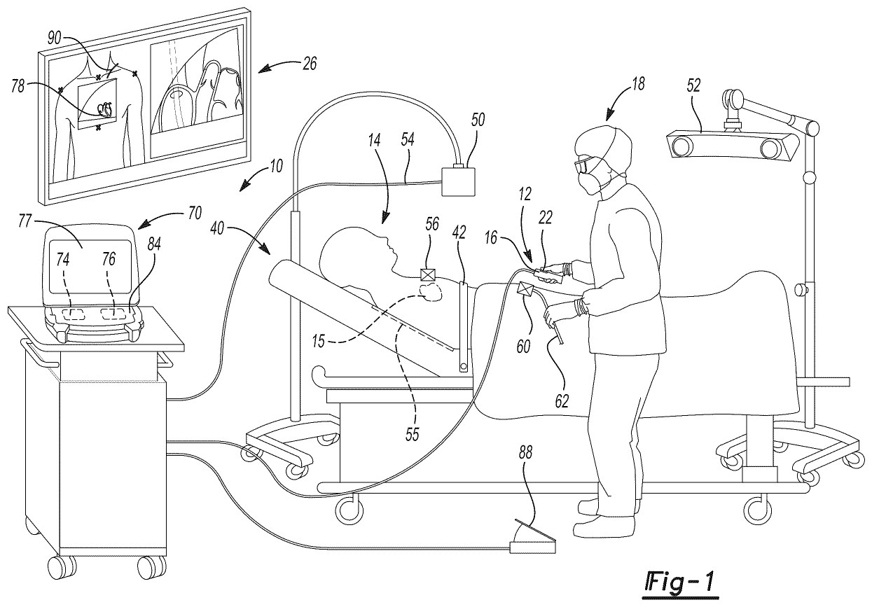

FIG. 1 is a diagram illustrating an overview of a navigation system 10 that can be used for various procedures. The navigation system 10 can be used to track the location of an item, such as an implant or an instrument, and at least one imaging system 12 relative to a subject, such as a subject 14 that may include a human patient and/or other living or non-living subject. It should be noted that the navigation system 10 may be used to navigate any type of instrument, implant, or delivery system, including: catheters, stylets, leads for cardiac rhythm management devices such as pacemakers, leadless pacemakers and delivery systems therefor, guide wires, arthroscopic systems, ablation instruments, stents, orthopedic implants, spinal implants, deep brain stimulation (DBS) probes, mechanical parts, Transesophageal Echocardiography (TEE), intra-cardiac echocardiography (ICE), etc. Non-human or non-surgical procedures may also use the navigation system 10 to track a non-surgical or non-human intervention of the instrument or imaging device. Moreover, the instruments may be used to navigate or map any region of the body. The navigation system 10 and the various tracked items may be used in any appropriate procedure, such as one that is generally minimally invasive or an open procedure.

The navigation system 10 can interface with or integrally include an imaging system 12 that is used to acquire pre-operative, intra-operative, or post-operative, or real-time image data of the patient 14. For example, the imaging system 12 can be an ultrasound imaging system (as discussed further herein) that has a tracking device 22 attached thereto. The tracking device 22 may be tracked with the tracking system to determine a pose of the imaging system 12. The pose may include an orientation, e.g., three or more degrees of orientation (e.g., yaw, pitch, and roll) and/or a position (e.g., three degrees of freedom in physical space (e.g., x-axis, y-axis, and z-axis). The tracking system 10 may further determine appropriate pose information regarding the tracking device 22. The pose of the imaging system 12 can then be determined based on the tracked pose of the tracking device 22. The imaging system 12 may be used to generate image data to provide images for viewing with a selected display device 26, which may any appropriate display device including an augmented and/or virtual reality display device worn/used by the user 18 such as those disclosed in U.S. Pat. App. Pub. No. US2018/0078316A1, incorporated herein by reference.

It will be understood any appropriate subject can be imaged and any appropriate procedure may be performed relative to the subject. The navigation system 10 can be used to track various tracking devices, as discussed herein, to determine locations of the patient 14. The tracked poses of the patient 14 can be used to determine or select images for display to be used with the navigation system 10. The initial discussion, however, is directed to the navigation system 10 and the exemplary imaging system 12.

In the example shown, the imaging system 12 includes an ultrasound (US) imaging system with an US housing 16 that is held by a user 18 while collecting image data of the subject 14. It will be understood, however, that the US housing 16 can also be held by a stand or robotic system while collecting image data. The US housing 16 and included transducer can be any appropriate US imaging system 12, such as the M-TURBO® sold by SonoSite, Inc. having a place of business at Bothell, Washington. Associated with, such as attached directly to or molded into, the US housing 16 or the US transducer housed within the housing 16 is at least one imaging system tracking device 22. The tracking device 22 may be any appropriate tracking device such as an electromagnetic tracking device and/or an optical tracking device. The tracking devices can be used together (e.g., to provide redundant tracking information) or separately. Also, only one of the two tracking devices may be present. It will also be understood that various other tracking devices can be associated with the US housing 16, as discussed herein, including acoustic, ultrasound, radar, electrical impedance, and other tracking devices. Also, the tracking device 22 can include linkages or a robotic portion that can determine a location relative to a reference frame.

In various embodiments, a secondary imaging system may also be presented and/or used to generated image data of the patient 14. The second imaging system may include an O-arm® imaging device sold by Medtronic Navigation, Inc. having a place of business in Louisville, Colorado, USA, a C-arm imaging system, or other appropriate imaging system. The second imaging system can include those disclosed in U.S. Pat. Nos. 7,188,998; 7,108,421; 7,106,825; 7,001,045; and 6,940,941; all of which are incorporated herein by reference. The second imaging system, however, need not be present and the imaging system 12, which may be only the US transducer, may be the only imaging system to generate images to be displayed. As discussed herein, various other image data may also be displayed such as an avatar 120 of the patient 14.

The imaging system 12 may be tracked, as discussed above. Various tracking systems may include and/or require calibration of the imaging system. Thus, the pose of the tracking device 22 relative to a plane of the US imaging system may be determined and/or known. Various systems and methods are disclosed in U.S. U.S. Pat. Nos. 6,379,302; 6,669,635; 6,968,224; 7,085,400; 7,831,082; 8,320,653; 8,811,662; and 9,138,204, and U.S. Patent Application Publication Nos. 2004/0097805 A1, 2019/0183577 A1, and 2021/0369394 A1, all of which are incorporated herein by reference.

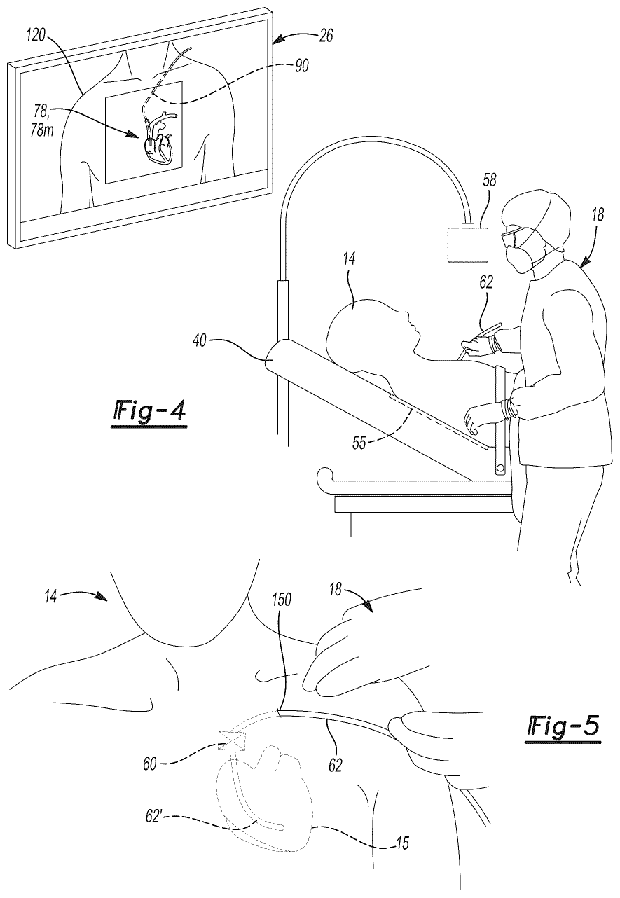

As illustrated in FIGS. 1, 3, and 4, the patient 14 can be fixed in a pose relative to a selected object, such as onto an operating table 40, but is not required to be fixed to the table 40. The table 40 can include a plurality of straps 42. The straps 42 can be secured around the patient 14 to fix the patient 14 relative to the table 40. Various apparatuses may be used to position the patient 14 in a static position on the operating table 40. Examples of such patient positioning devices are set forth in commonly assigned U.S. patent application Ser. No. 10/405,068, published as U.S. Pat. App. Pub. No. 2004/0199072 on Oct. 7, 2004, entitled “An Integrated Electromagnetic Navigation And Patient Positioning Device”, filed Apr. 1, 2003 which is hereby incorporated by reference. Other known apparatuses may include a Mayfield® clamp.

The navigation system 10 includes at least one tracking system. The tracking system can include at least one localizer. In one example, the tracking system can include an electromagnetic (EM) localizer 50. The tracking system can be used to track instruments relative to the patient 14 or within a navigation space. The navigation system 10 can use image data from the imaging system 12 and information from the tracking system to illustrate locations of the tracked instruments, as discussed herein. The tracking system can also include a plurality of types of tracking systems including an optical localizer 52 in addition to and/or in place of the EM localizer 50. When the EM localizer 50 is used, the EM localizer can communicate with or through a localizer communication 54 that may be wired or wireless. The EM localizer 50 may also include and/or take the form of an alternative pad or flat EM localizer 55.

The optical localizer 52 and the EM localizer 50 can be used together to track multiple instruments or used together to redundantly track the same instrument. Various tracking devices, including those discussed further herein, can be tracked and the information can be used by the navigation system 10 to allow for an output system to output, such as a display device to display, a position of an item. Briefly, tracking devices can include a patient or reference tracking device 56 to track the patient 14, an instrument tracking device 60 to track an instrument 62, and/or other appropriate tracking devices for one or more portions. Patient or reference tracking device 56 and instrument tracking device 60 may include those disclosed in U.S. Pat. Nos. 8,060,185 and 8,644,907, both incorporated herein by reference. The tracking devices allow selected portions of the operating theater to be tracked relative to one another with the appropriate tracking system, including the optical localizer 52 and/or the EM localizer 50. The reference tracking device 56 can also or alternatively be positioned on an instrument and positioned within the patient 14, such as within a heart 15 of the patient 14.

It will be understood that any of the tracking devices 22, 56, 60 can be optical or EM tracking devices, or both, depending upon the tracking localizer used to track the respective tracking devices. It will be further understood that any appropriate tracking system can be used with the navigation system 10. Alternative tracking systems can include radar tracking systems, acoustic tracking systems, ultrasound tracking systems, electrical impedance tracking systems, radio frequency beacon, and the like. Exemplary tracking systems include those disclosed in U.S. Pat. Nos. 7,676,268; 8,532,734; and 8,494,608, all incorporated herein by reference. Each of the different tracking systems can be respective different tracking devices and localizers operable with the respective tracking modalities. Also, the different tracking modalities can be used simultaneously as long as they do not interfere with each other (e.g., an opaque member blocks a camera view of the optical localizer 52).

An exemplary EM tracking system can include the STEALTHSTATION® AXIEM™ Navigation System, sold by Medtronic Navigation, Inc. having a place of business in Louisville, Colorado. Exemplary tracking systems are also disclosed in U.S. Pat. No. 7,751,865, issued Jul. 6, 2010 and entitled “METHOD AND APPARATUS FOR SURGICAL NAVIGATION”; U.S. Pat. No. 5,913,820, titled “Position Location System,” issued Jun. 22, 1999 and U.S. Pat. No. 5,592,939, titled “Method and System for Navigating a Catheter Probe,” issued Jan. 14, 1997, all herein incorporated by reference.

Further, for EM tracking systems it may be necessary to provide shielding or distortion compensation systems to shield or compensate for distortions in the EM field generated by the EM localizer 50. Exemplary shielding systems include those in U.S. Pat. No. 7,797,032, issued on Sep. 14, 2010 and U.S. Pat. No. 6,747,539, issued on Jun. 8, 2004; distortion compensation systems can include those disclosed in U.S. patent Ser. No. 10/649,214, filed on Jan. 9, 2004, published as U.S. Pat. App. Pub. No. 2004/0116803, all of which are incorporated herein by reference.

With an EM tracking system, the EM localizer 50 and the various tracking devices can communicate through an EM controller. The EM controller can include various amplifiers, filters, electrical isolation, and other systems. The EM controller can also control the coils of the EM localizer 50 to either emit or receive an EM field for tracking. A wireless communications channel, however, such as that disclosed in U.S. Pat. No. 6,474,341, entitled “Surgical Communication Power System,” issued Nov. 5, 2002, herein incorporated by reference, can be used as opposed to being coupled directly to the EM controller. The EM controller can be incorporated into a navigation processing system 70.

It will be understood that the tracking system may also be or include any appropriate tracking system, including a STEALTHSTATION® TRIA®, TREON®, and/or S7™ Navigation System having an optical localizer, similar to the optical localizer 52, sold by Medtronic Navigation, Inc. having a place of business in Louisville, Colorado. Further alternative tracking systems are disclosed in U.S. Pat. No. 5,983,126, to Wittkampf et al. titled “Catheter Location System and Method,” issued Nov. 9, 1999, which is hereby incorporated by reference. Other tracking systems include an acoustic, radiation, radar, electrical impedance, etc. tracking or navigation systems.

The navigation system 10 can include a navigation processing unit or module 74 that can communicate or include a navigation memory 76, which may be included in the navigation processing system 70. The navigation processing system 70 may further include a display device 77. The navigation processing unit 74 can include a processor (e.g., microprocessor, a central processing unit, etc.). In various embodiments, the navigation processing unit may execute instructions to determine one or more poses of the tracking devices based on signals from the tracking devices. The navigation processing unit 74 can receive information, including image data, from the imaging system 12 and tracking information from the tracking systems, including the respective tracking devices and/or the localizers 50, 54. Image data can be displayed as an image 78 on the display device 26. The display device may be separate from and/or integrated into the navigation system, thus, the display device 26 may include the display device 77. The navigation system 70 can include appropriate input devices, such as a keyboard 84. It will be understood that other appropriate input devices can be included, such as a mouse, a foot pedal 88 or the like which can be used separately or in combination. Also, all of the disclosed processing units or systems can be a single processor module (e.g., a single central processing chip) that can execute different instructions to perform different tasks.

An image processing unit or module can process image data from the imaging system 12 and a separate first image processor (not illustrated) can be provided to process or pre-process image data from the imaging system 12. The image data from the image processor can then be transmitted to the navigation processor 74. It will be understood, however, that the imaging systems need not perform any image processing and the image data can be transmitted directly to the navigation processing unit 74. Accordingly, the navigation system 10 may include or operate with a single or multiple processing centers or units that can access single or multiple memory systems based upon system design.

In various embodiments, the imaging system 12 can generate image data that may be used to compose the image 78 and define an image space that can be registered to a patient space or navigation space that is defined by and/or relative to the patient 14. In various embodiments, the position of the patient 14 relative to the imaging system 12 can be determined by the navigation system 10 with the patient tracking device 56 and the imaging system tracking device(s) 22 to assist in and/or maintain registration. Accordingly, the position of the patient 14 relative to the imaging system 12 can be determined.

Manual or automatic registration of the image space to the subject space can occur. In various embodiments, the registration can occur by matching fiducial points in image data with fiducial points on the patient 14. Registration of image space to patient space allows for the generation of a translation map between the patient space and the image space. According to various embodiments, registration can occur by determining points that are substantially identical in the image space and the patient space. The identical points can include anatomical fiducial points or implanted fiducial points. Exemplary registration techniques are disclosed in U.S. patent application Ser. No. 12/400,273, filed on Mar. 9, 2009, now published as U.S. Pat. App. Pub. No. 2010/0228117 and in U.S. Pat. No. 9,737,235, issued Aug. 22, 2017, both incorporated herein by reference.

According to various embodiments, the imaging system 12 can be used with an un-navigated or navigated procedure. In a navigated procedure, a localizer and/or digitizer, including either or both of an optical localizer 52 and/or an electromagnetic localizer 50, 55 can be used to generate a field and/or receive and/or send a signal within a navigation domain relative to the patient 14. The navigated space or navigational domain relative to the patient 14 can be registered to the image 78. Correlation, as understood in the art, is to allow registration of a navigation space defined within the navigational domain and an image space defined by the image 78. The patient tracker or dynamic reference frame 56 can be connected to the patient 14 to allow for a dynamic registration and maintenance of registration of the patient 14 to the image 78.

Once registered, the navigation system 10 with or including the imaging system 12, can be used to perform selected procedures. Selected procedures can use the image data generated or acquired with the imaging system 12. Further, the imaging system 12 can be used to acquire image data at different times relative to a procedure. As discussed herein, image data can be acquired of the patient 14 prior to the procedure for collection of automatically registered image data or cine loop image data. Also, the imaging system 12 can be used to acquire images for confirmation of a portion of the procedure. Thus, image data may be acquired at any appropriate time and may be registered to the patient 14.

Registration and navigated procedures are discussed in U.S. Pat. No. 8,238,631, incorporated herein by reference. Upon registration and tracking of the instrument 62, a graphic representation 90 (e.g., an icon, indicium, animation or other or visual representation) may be displayed relative to, including overlaid (e.g., superimposed) on, the image 78. The graphic representation can be generated using any suitable software and/or hardware, including within limitation the processing unit 74. The instrument 62 could incorporate one or more electromagnetic coil sensors along a portion of its length (e.g., the portion to be rendered in the graphic representation.) In lieu of or in addition to electromagnetic coil sensor(s), a shape sensing fiber (e.g. one made up of several fiber Bragg gratings) could be employed. An algorithm that renders the pose, curve and/or shape of instrument 62 could be employed, e.g. using the flexure formula to take the known bending characteristics of a particular instrument 62 into account; the pose of the sensor(s) (e.g. electromagnetic sensor(s), etc.) determined and the resulting curve/pose/shape of the instrument 62 generated. The pose, shape and/or position of the rendered portion of the instrument 62 can be displayed and, if desired, labeled. Alternatively, instead of or in addition to using the processing unit 74, the graphical representation can be captured in the ultrasound image and provided to the processing unit. The image 78 may be an appropriate image and may include one or more 2D images, such as 2D images that are acquired at different planes. Images may also be a 3D image, or any appropriate image as discussed herein.

In addition to registering the subject space to the image space, however, the imaging plane of the US imaging system 12 can also be determined. By registering the image plane of the US imaging system 12, imaged portions can be located within the patient 14. For example, when the image plane is calibrated to the tracking device(s) 22 associated with the US housing 16 then a position of an imaged portion of the heart 15, or other imaged portion, can also be tracked as disclosed in U.S. Pat. Nos. 6,379,302; 6,669,635; 6,968,224; 7,085,400; 7,831,082; 8,320,653; 8,811,662; and 9,138,204 all of which are incorporated herein by reference.

Once the patient 14 is in condition for a procedure, the patient 14 may be positioned on the table 40, as noted above. The display 26 and/or the display 77 may display various information regarding the patient 14 and/or other information selected by the user 18. As noted above, the display 26 may illustrate the image 78 that may be acquired with the imaging system 12. The image 78 may also be displayed on the display 77. Also as noted above, the instrument 62 may be tracked and the graphic representation 90 of the instrument 62 may be displayed on the display 26, such as relative to the image 78. In addition and/or alternatively, the graphic representation 90 may also be displayed relative to a patient avatar 120.

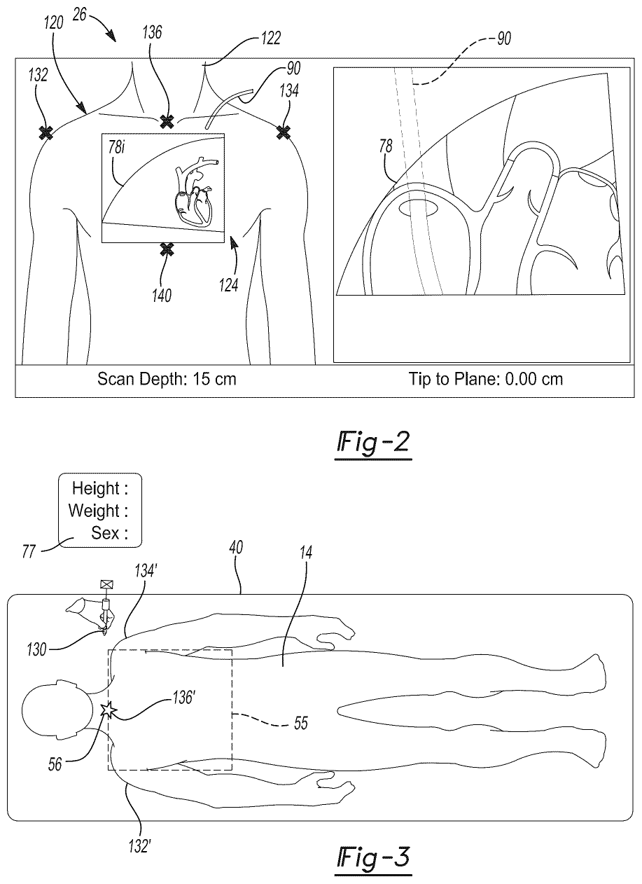

The patient avatar 120 may be illustrated on a selected display, as illustrated in FIG. 2, such as the display 26. The patient avatar 120 may be based on a general avatar that includes various features of the patient 14, as noted herein, but is sized to the current and specific patient. The avatar 120 may be illustrated as a two-dimensional (2D) image and/or a three-dimensional (3D) image. The avatar 120 may be any appropriate shape to assist in identifying various positions and relative positions of portions of the patient 14. The avatar 120, according to various embodiments, is sized to the specific patient as disclosed herein.

The patient avatar 120 may generally illustrate a portion of the patient 14 and may be sized relative to the patient 14. For example, the patient avatar may illustrate an inferior portion of a neck 122, a pectoral region 124, and other appropriate regions of the patient 14. The patient avatar 120 may be displayed on the display device 26 to illustrate a general representation of the patient 14 without requiring an image acquisition of the same portion of the patient 14. Therefore, the patient avatar 120 may provide a general roadmap or indication of the position of various tracked portions, as discussed further herein. The avatar 120 may however, as discussed herein, be morphed to match a relative size of the patient 14 to provide an indication of relative anatomical portions of the patient 14 and a pose of the tracked portions, such as the instrument 62, relative thereto.

With continuing reference to FIG. 2 and additional reference to FIG. 3, the avatar 120 may be generally sized to the patient 14 according to one or more appropriate procedures. The avatar 120 may be sized or morphed in real time to match relative dimensions of the patient 14. The relative positions of the anatomic portions may include a relative position of a suprasternal notch and an exterior shoulder or inferior jugular vein, or any other suitable anatomical location(s).

In various embodiments, for example, the user 18 may provide or include information regarding the patient 14. As noted above the navigation processing system 70 may include various inputs that allow the user 18 to input information. Therefore, as illustrated in FIG. 3, the user 18 may input information such as a height, a weight, and a sex of the patient 14. The input information is used by the processor module 74 to execute selected instructions to size the patient avatar 120 to the actual patient 14. The resizing of the avatar 120 may be based upon recalling of a selected or known predetermined size from a database, reconfiguring or resizing the patient avatar 120 based upon the input information, or other appropriate information. Therefore, the avatar 120 may be sized to substantially match the patient 14 without acquiring an image of the patient 14 for display on the display device 26.

In addition or alternatively thereto, the user 18 may also identify or size the avatar 120 to the patient 14 with a tracked indicator or tracked member 130. For example, the tracked indicator 130, which may be an indicator or probe usable to touch a surface of the patient 14, may be moved by the user 18 to various predetermined positions on the patient 14 as indicated on the avatar 120. The tracked indicator 130 may be tracked with any appropriate tracking system, including those discussed herein according to various embodiments such as the localizer system of U.S. Pat. No. 5,983,126, hereby incorporated by reference. The user 18 may move the tracked indicator 130 to various points, such as the exterior shoulder portions 132′, 134′ as illustrated by the icons 132 and 134. Additionally, the user 18 may move the tracked indicator 130 to other portions such as a suprasternal notch 136 and a sternum 140 of the patient 14 or outer boundary positions. It is understood that the tracked indicator 130 may be moved to any appropriate position and the above-noted positions are merely exemplary. Nevertheless, based upon the tracked position of the tracked indicator 130, the avatar 120 may be sized to morph the identified points to a representation of the patient 14 for display on the display device 26. Also, the points on the avatar 120 may be registered to the patient 14 by identifying them in the patient space relative to the patient 14 and identifying them on the avatar 120.

The avatar 120 may also be morphed to actual measurements taken of the patient 14. For example, a distance between the shoulder points 132′, 134′ on that the patient 14 may be measured, such as with a tape measure. The measured distance may be input within the navigation processing system at 77. The avatar 120 may be sized based upon the measurements.

Regardless of the technique, the avatar 120 may be matched or morphed to substantially mimic the shape of the patient 14. The avatar 120, as discussed further herein, may then be used as an illustration of a pose of the tracked instrument, such as the instrument 62, during a procedure on the patient 14. Thus, the avatar 120 may provide a representation of a position of the tracked instrument 62 without requiring an image acquisition of the patient 14, such as with a fluoroscopic imaging system. The avatar 120, as it is morphed to the specific patient may or will be displayed as a patient specific avatar. Thus, the avatar 120 may include general information, but is displayed and registered to the patient 14 as a patient specific avatar 120 including relevant locations and relative positions of various portions of the patient 14. The specific patient avatar illustrates the relative positions of the anatomy of the patient 14 that is the current patient. For example, the position of the heart 15 relative to the suprasternal notch.

The avatar 120 may be registered to the patient 14 in any appropriate manner, including those discussed above. In various embodiments, for example, when the tracked indicator 130 is tracked to determine positions of the patient 14 for marking or changing the avatar 120. The patient 14 after the various points are determined in both the patient 14 and the avatar 120 may be registered thereto. Additionally, and/or alternatively, the patient tracker 56 may be positioned on the patient 14 at an appropriate position. The position of the patient tracker 56 may be at the suprasternal notch 136′ which relates to a point on the avatar 120, such as the suprasternal notch 136. The position of the suprasternal notch 136 in the avatar 120 may be determined or known to be the position of the patient tracker 56 to allow for registration of the avatar 120 relative to the patient 14. Also, the patient tracker 56 may maintain registration even when the patient 14 moves relative to the localizer(s). It is understood that any appropriate number of the patient trackers 56 may be utilized, including each may be used for registration and/or more than one of the patient trackers 56 may be used.

The localizers, including the EM localizer 58, may also or alternatively be fixed or positioned relative to the patient 14 in an appropriate manner. For example, the localizer 58 may be fixed relative to the table 14 such that it is immovable during the procedure. Thus, the field generated with the EM localizer 58 can be at a known position relative to the patient 14 and allow for a registration of the avatar 120 to the patient 14 based upon the known position of the EM localizer 58 relative to the patient 14. The localizer 55 may also be positioned under the patient 14, such as between the patient 14 and the bed 40. Localizer 55 may include field generating portions, such as coils, that generate a field relative to the patient 14. The flat or panel localizer 55 may be positioned at a known position relative to the patient 14, such as aligning the shoulder edges 132′, 134′ to known or predetermined portions of the panel localizer 55. As the panel localizer 55 is at a known position relative to the patient 14, and the avatar 120 is morphed to the patient and 14, the avatar 120 may be registered to the patient 14 due to the position of the panel localizer 55.

Regardless, the avatar 120 may be registered to the patient 14. Registration of the avatar 120 relative to the patient 14 allows the avatar 120 to be used to assist navigation and guiding of the instrument 62, or any appropriate instrument relative to the patient 14. As the tracked position of the instrument 62 is determined with the navigation system 10, the pose of the instrument 62 may be illustrated with a graphical representation, such as the graphic representation 90 relative to the image 78 and/or the avatar 120.

As illustrated in FIGS. 2-5, the instrument's graphic representation 90 is displayed superimposed on the avatar 120. The user 18 may view the display 26 and understand the tracked position of the instrument 62 relative to the patient 14 by viewing the graphic representation 90 of the instrument 62 in the avatar 120 on display 26. The user 18 may then understand the relative position of the instrument 62 to the patient 14 and determine that the instrument 62 is generally moving and/or is positioned in a selected or procedure planned position. The user 18, thus, may understand this by viewing the display 26 and the avatar 120 without requiring additional or any image data being acquired of the patient 14.

In various embodiments, for example, image data may be acquired of the patient 14. As noted above, the imaging system 12 may acquire image data of the patient 14. The imaging system 12 may generally be a non-ionizing imaging system, such as an ultrasound imaging system. The ultrasound image may generally be collected at a single plane relative to the housing 16. The US imaging system 12 with the tracker 22 may be tracked relative to the patient 14 so that the image may also be registered to the patient 14. Thus, the image 78 may also be displayed superimposed on the avatar 120, such as the image superimposition 78i. The image may also be displayed separately from the avatar, as also illustrated in FIG. 2.

As the image 78 may be registered to the patient 14, a single image acquisition may occur and the image 78 may be displayed for the procedure. Further, as the image 78 is registered to the patient the graphic representation 90 of the instrument 62 may be displayed at the tracked pose on the image 78. Thus, the US imaging system 12 may be used at one point in time to acquire the image data, but it may be used to illustrate the selected portion of the patient 14, such as the heart, for an entire procedure. A constant imaging of the patient 14 is not required to illustrate or know a position of the instrument 62 as the instrument 62 is tracked and the image 78 is registered to the patient 14 due to the US tracker 22.

In a procedure, once the avatar 120 and/or the image 78 are registered to the patient, the user 18 may perform the procedure on the patient 14 with the tracked instrument(s). The procedure may be any appropriate procedure, such as placing a pacing lead and/or a catheter to assist in placing the lead. Other appropriate procedures may also be performed.

In various embodiments, the user 18 may form an incision, such as an incision 150 on the patient 14, and move the instrument 62 into the patient 14 through the incision 150. The tracked portion 60 on the instrument 62 may be tracked so that a position of the instrument 62 may be displayed relative to the avatar 120 and/or the image 78, as noted above. The instrument 62 may then be moved relative to the patient 14. In various embodiments, for example, the instrument 62 may be a catheter and/or lead for an implant, or a stylet or a delivery system for an implant such as a leadless pacemaker or a stent. As is understood by one skilled in the art, an implant, including an electrode, may be positioned relative to one or more portions of the heart 15 of the patient 14.

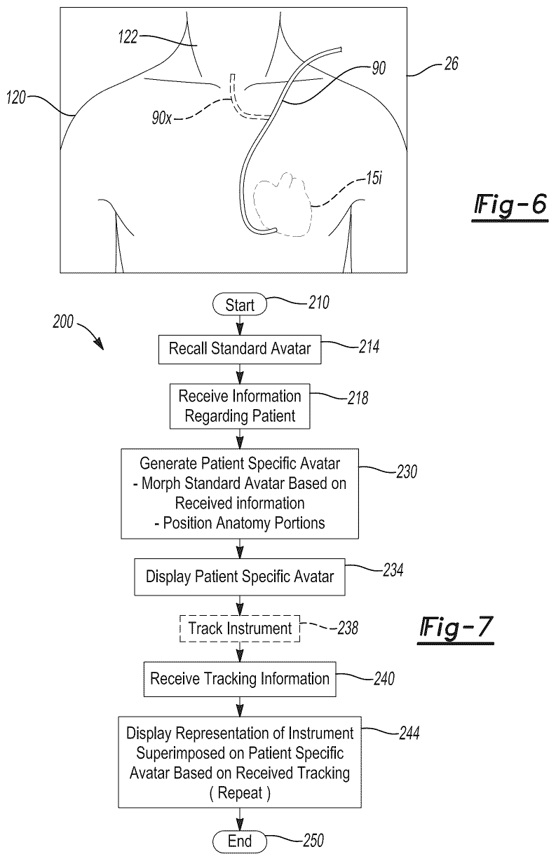

With continuing reference to FIG. 5 and additional reference to FIG. 6, the position of the instrument 62 may be illustrated with the graphic representation 90 over time and/or instantaneously relative to the avatar 120. The graphic representation 90 illustrates the pose of the instrument 62 tracked relative to the patient 14. The graphic representation 90 may be viewed by the user 18 to understand a real-time position of the instrument 62 within the patient 14 via the avatar 120. Again, the avatar 120 may be generated without requiring image data of the patient 14, such as with fluoroscopy.

The avatar 120 may also assist the user 18 in identifying or determining an orientation of the instrument 62 as being in a selected or in a not-selected pose relative to the patient 14. For example, as illustrated in FIG. 6, the avatar 120 may be viewed by the user 18 during a procedure. The user 18 may view the representation 90 of the instrument 62 as it moves in a selected direction and/or position. The tracked pose of the instrument 62 may be updated in substantially real time and displayed relative to the avatar 120. Therefore, the graphic representation 90 of the instrument 62 when it is moving in a not-selected position or and/or pose may also be illustrated and understood by the user 18. As illustrated in FIG. 6 a representation 90x may illustrate movement and/or pose toward a neck 122 of the patient 14. This may be a not-selected pose when the instrument 62 is selected to be moving toward the heart 15 of the patient 14. Therefore, the user 18 may view the graphic representation 90x and understand that the instrument 62 is moving away from the heart 15 of the patient 14. A representation of the heart 15 may also be displayed on the avatar 120 including the image 78 and/or with a heart representation 15a that is included in the avatar 120 and appropriately located with the patient specific information.

Thus, the user 18 may view and understand the position of the instrument 62 during the placement of an implant, which may include, for example, a stimulation lead for a selected system. The lead may include a pacing electrode be for a cardiac stimulation system, such as the AZURE®, ADAPTA®, or other appropriate heart pacemaker or defibrillation systems sold by Medtronic, Inc. having a place for business in Minnesota, USA.

Turning reference to FIG. 7, a method 200 for determining and displaying a patient specific avatar is illustrated. The process may begin in Block 210 and include recalling a standard avatar in Block 214. In recalling a standard avatar the processor, such as the processor 74, may recall a standard avatar including various anatomical features, including exterior dimensions of a patient's anatomy and selected internal anatomy such as a heart, physical location of the heart, jugular vein, or the like.

Information regarding the patient, such as the specific patient, may be received in Block 218. The information regarding the patient may be any appropriate information, including that discussed above. Therefore, the information may include general dimensions of the current patient, including a height, weight, sex, or the like. Information received in Block 218 may also include specific tracked points of predetermined portions (e.g., shoulder edges) of the patient 14. Further the received information may include measurements of the patient. Regardless, the received information of the patient may be patient specific information regarding the patient for a current or real-time procedure.

A patient specific avatar is then generated in Block 230. The generation of a patient specific avatar may include morphing or fitting the standard avatar based upon the received information. In morphing the standard avatar, the method may include altering dimensions of the standard avatar based upon a ratio given the information received regarding the current patient. The ratio may be based upon the known dimensions of the current patient based upon known or average dimensions of various anatomical portions relative to one another based upon the dimensions of the specific patient. This may also include having predetermined average positions and relative positions of various anatomical features. In addition and/or alternatively thereto, the tracked position of the instrument may identify specific anatomical locations. The standard avatar may then be morphed based upon the tracked specific locations for the specific patient. Thus, the avatar may be morphed based upon selected instructions including an algorithm to generate the patient specific avatar in Block 230.

After generating the patient specific avatar Block 230, the patient specific avatar may be displayed in Block 234. As discussed above, the display 26 may display the patient specific avatar for selected procedures. The display of the patient specific avatar may allow the user 18 to identify and understand relative positions or locations of various portions of the current patient 14.

An instrument may then be tracked, optionally and according to various embodiments, in Block 238. Although the tracking of the instrument is an optional part of the method 200, such tracking may allow for information regarding a pose of an instrument to be determined. Regardless of whether the instrument is tracked, the method 200 may include receiving tracking information in Block 240.

Receiving tracking information in Block 240 may include tracking one or more tracking devices to determine a pose of various elements or portions, such as the pose of the instrument 62 via the instrument tracker 60 and/or the patient 14 via the patient tracker 56. The patient specific avatar may be registered to the patient based upon the known location of one or more specific portion(s) of the patient, such as the positioning of the patient tracker 56 in the suprasternal notch of the patient 14. The suprasternal notch of the avatar 120 may then be registered for determination of the pose of the instrument 62 relative to the patient tracker 56. Therefore, the pose of the instrument 62 may be displayed on the display device 26 such as the superimposed graphic representation 90 on the avatar 120.

Thus, the pose of the instrument may be superimposed on the avatar 120 in Block 244. Again, this allows a user 18 to view and understand a pose of the tracked instrument 62 on a representation of the patient 14 without requiring image data to be acquired to the patient 14. While image data of the patient 14 may be acquired, such as with the US imaging system 12, such image information is not required. The image data information from the US imaging system 12 may assist in finalizing or determining a specific location of the instrument 62 relative to a selected portion, such as a specific portion of the heart 15 of the patient 14. However, the position of the instrument 62 may be determined without the image data. Thus, the user may understand a position of the instrument 62, including a movement of the instrument 62 toward a selected position, such as the RV septum of the patient 14.

The method 200 may then end in Block 250. And ending the method 250 it is understood that the method 200 may be repeated as often as necessary or selected to allow for up to date and/or real-time tracking and illustration of a pose of the instrument 62 relative to the avatar 120. Thus, the method 200 may be repeated at a selected rate and for a selected period, such as selected by the user 18.

As noted above, moving the instrument 62 relative to the patient 14, such as relative to the heart 15, may be accomplished while tracking a pose of the instrument 62. As noted above, tracking a pose of the instrument 62 can involve attempting to determine a physical location and/or orientation of the instrument 62 in the physical space. The tracked posed may include a location which includes X, Y, and Z axis positions and orientation which may include yaw, pitch, and roll orientations in space. The physical space may also be referred to as navigation space and is generally defined relative to the patient 14 and/or the heart 15. The patient tracker 56 allows the pose of the patient 14, including the heart 15, to be tracked. Tracking a pose relative to a portion of the patient 14, including the heart 15, may include systems such as those disclosed in U.S. Pat. No. 7,697,972 issued Apr. 13, 2010; U.S. Pat. No. 8,046,052 issued Oct. 25, 2011; U.S. Pat. No. 8,180,428 issued May 15, 2012; and U.S. Pat. No. 8,663,120 issued Mar. 4, 2014, all of which are herein incorporated by reference.

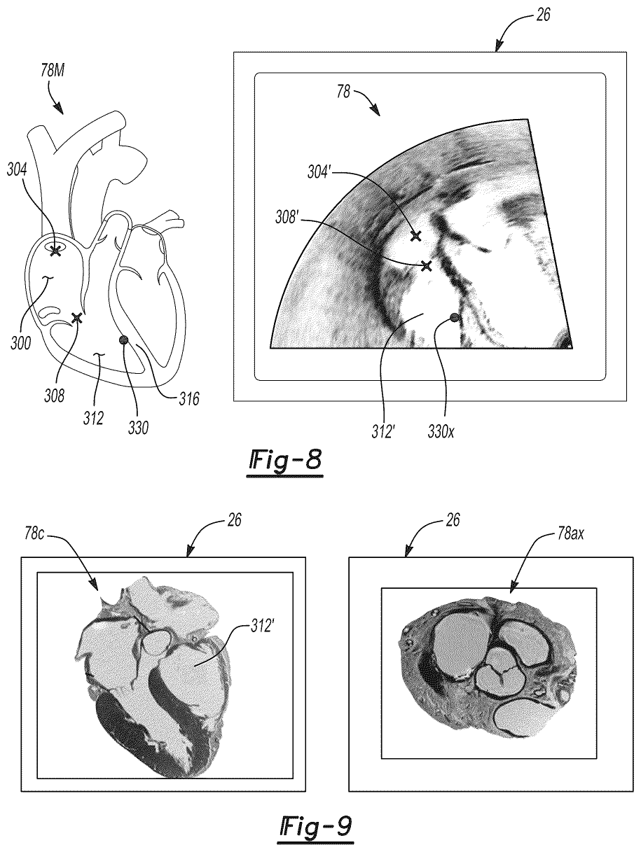

With continuing reference to FIGS. 1-7, and additional reference to FIG. 8, the instrument 62 may be tracked relative to the patient 14 which may be displayed such as within the graphic representation 90 relative to selected portions including the avatar 120, the image 78, and/or an atlas 78m, as illustrated in FIG. 8. The atlas 78m may be based upon the selected information, such as based on a plurality of images of patients that have been averaged and portions thereof identified. For example, the atlas 78m may be a model based on the image data from a plurality of images. The atlas 78m (also referred to as a model) may have selected portions therein that are identified. In various embodiments, the atlas 78m may include the following identified locations: a right ventricle 300, a superior vena cava ostium 304, a tricuspid valve 308, a right ventricle 312, and a right ventricle septum 316.

With reference to FIG. 8, the image 78 is an example of one image in a set of images that can be used to build the atlas 78m. The atlas 78m may also be generated using the patient's pre-procedure imaging like a CT or MRI. The image 78 may be obtained with a selected imaging system, including the US imaging system 12, as discussed above. A patient specific anatomy model may be generated using multiple 2D/3D views/planes stitched together, for example to reconstruct the endocardium of the heart. To facilitate building the atlas 78m, a clinician may identify various relevant portions or locations within the image 78. The user 18 may identify portion(s) or location(s) in the image 78 shown in the right-hand side of FIG. 8 as the superior vena cava entrance 304′, the tricuspid valve 308′, and/or the right ventricle 312′. Other locations may include endocardium or blood pool boundary may include the identification of the portions/locations within the heart. The location identification may be done manually by the user 18 and/or with assistance of an automatic location identification, such as segmenting a valve, etc.

Multiple images similar to the image 78, e.g., multiple similar images from a population of patients other than the patient 14, with similar identified portion(s) or location(s) may be combined to build the atlas 78m to illustrate the identified portion(s) or location(s) on a model of the imaged anatomy or heart, which model represents a typical or average patient anatomy. Alternatively, the atlas 78m may be built only from images of the anatomy of the patient 14, and the portion(s) or location(s) of interest applied to the atlas based on identification within the images of the patient's own anatomy, and/or based on a compilation of images from other patients' anatomy and an estimated and/or average position of the portion(s) or location(s) of interest within the compilation of images. The atlas 78m may also be morphed to actual measurements taken of the patient 14.

Based upon the user's 18 identification in the image 78 of the various portions/locations, which may include all of or selected number of portions/locations, a processing module or unit, such as the processor 74, may execute selected instructions to register or identify various portions in the image 78 and/or on the atlas 78m for viewing by the user 18. For example, in the atlas 78m the right ventricle septum may be identified as a selected point including a target 330. The image 78 may also, therefore, have the target identified thereon 330x to illustrate a proposed or selected position of the right ventricle septum (RV septum), e.g., to serve as a target for delivery of a catheter, stylet and/or pacing lead, or a leadless pacemaker. The predetermined portions in the model 78m, therefore, may be used to assist in identifying portions of the image 78 based on one or more portions identified by the user 18 and/or appropriate image identification systems. Therefore, the model 78m may be used to assist in identifying portions within the image 78 to assist in the procedure. The user may view the image 78 and identify or evaluate the target 330x prior to and/or during the procedure.

The image 78, as discussed above, may also include a plurality of images that are generated with the imaging system 12. The US imaging system 12 may acquire images of the patient 14, including the heart 15, in a plane. As the US housing 16 is moved, the plane of the image relative to the patient 14 may also change. Thus, the image 78 may include a plurality of images that are illustrated in one or more planes. For example, with reference to FIG. 9, schematic images that may represent an approximately coronal to the body co-ordinate system image 78c may be acquired and displayed and/or an image that is approximately axial to the body co-ordinate system 78ax may be displayed. The two images 78ap and 78ml may both be displayed and registered to the patient 14, as discussed above. Further, the tracked position of the instrument 62 may be displayed relative to one or both of the images 78ml and 78ap. Also, both of the images 78ml and 78ap may assist in registering or relating of the images to the model 78m. Additionally, the user 18 may view the images, as discussed above, and identify portions or locations thereon and of the model 78m that may be used to assist in identifying various other portions. For example, the user 18 may identify the tricuspid valve 308′ and the superior vena cava entrance 304′ and the model 78m may be used to identify various other portions or locations, such as the target 330x on the RV septum 330.

In addition to providing more than one view of the heart 15 of the patient 14, the two images 78ap and 78ml may also assist in registration to the model 78m. Thus, the identification of anatomy in the images 78 and/or one or both of the images and 78ap and 78ml, may be assisted by the registration of more than one image relative to the model or atlas 78m. As discussed above, the ultrasound imaging system 12 is registered to the patient 14, thus the images acquired with of the imaging system 12 may also be registered to the patient 14. The registration of the images to the patient may be based upon the tracking of the imaging system 12, such as with the imaging system tracking device 22 and the tracking of the patient 14, such as with the patient tracking device 56. Once the image 78 is registered to the patient 14, the instrument 62 may be tracked relative to the patient 14 and its pose may be displayed relative to the image 78. As discussed above, the pose of the instrument 62 may also be displayed relative to the avatar 120. The graphic representation 90 allows the user 18 to view the pose of the instrument 62 relative to both of the image 78 and the avatar 120.

Further, the instrument 62 may be tracked in real time. Therefore, the instrument may be displayed relative to the avatar 120 and/or the image 78 to provide a real time graphical representation of the instrument 62 relative to the heart 15, based upon a graphical representation displayed relative to the image 78. In addition to viewing the tracked pose of the instrument 62, the graphical representation may show the pose of the instrument 62 over time. Therefore, the graphic representation 90 may illustrate or provide a sense of an entire path of the instrument 62 as it moves from the incision 150 in the patient 14 to the current location within the patient 14. Further, the graphic representation 90 may provide a real-time feedback to the user 18 of a movement of the instrument 62.

In various embodiments, the images 78 may be acquired or based upon image data acquired at a selected time. For example, prior to the incision 150, image data may be acquired with the imaging system 12 to generate the image 78. Image 78 may thus be registered to the patient 14 and displayed with a display device 26 either alone and/or superimposed on the avatar 120. Nevertheless, the image 78 may not be a real time or current image and may be a static image. The instrument 62, however, may be tracked in real time and provide a current determination of the pose of the instrument 62. The graphic representation 90 may, therefore, be updated in real time and at a selected rate to illustrate a current position of the instrument 62 and/or a current shape or condition of the patient anatomy. The display of the graphical representation may also be updated in a selected rate to provide a perception of motion of the instrument 62. For example, a refresh of the pose of the instrument 62 and of the graphic representation 90 may be 10 times a second, 30 times a second, or any appropriate rate such as about one time a second to about 120 times per second. Thus, the pose of the instrument 62 may be illustrated to show a motion within the display device 26.

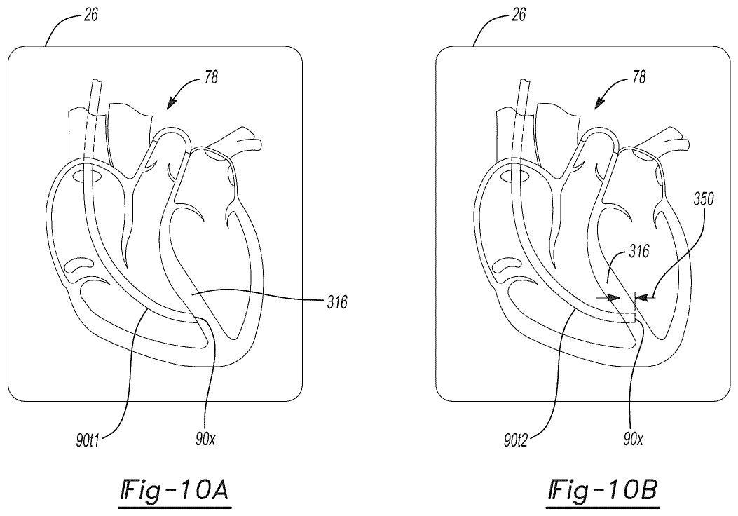

With reference to FIGS. 10A and 10B, the image 78 may be displayed on the display device 26. The image 78 may not change over time as the image 78 may be a static image that is acquired at a selected time, such as prior to positioning the instrument 62 within the patient 14. Nevertheless, the tracking device 60 may be tracked in real time on the device 62. Thus, as illustrated in FIG. 10A, the graphic representation 90 may include a first or initial time graphic representation 90t1. The graphic representation 90t1 may represent a first graphic representation at a selected time, that may be arbitrarily referred to as a first or initial time. After a selected period of time, a second graphic representation 90t2 may be displayed on the display device 26, as illustrated in FIG. 10B.

The graphic representation 90t2 may be displayed or have a selected position that is displaced a certain distance of 350 relative to the first graphic representation 90t1. The distance 350 may be any appropriate distance and may be based upon the various physical features, such as movement of a beating heart. The heart 15 of the patient 14 may beat (i.e., move due to the heartbeat), as is understood by one skilled in the art. Therefore, the graphic representation 90t2, including a terminal end 90x, may be shown in different positions based upon movements of the instrument 62. For example, as one skilled in the art will understand, when the terminal end of the instrument 62 contacts the RV septum 316, the terminal end of the instrument 62 may move due to the beating of the heart 15. Therefore, even if the user 18 is not moving the instrument 62, the instrument 62 may appear to move in real time. Real-time tracking of the instrument 62 with the tracking device and 60 may allow for a representation of the movement of the instrument 62 to be displayed on the display device 26. As discussed above, the movement of the instrument may be updated and displayed at any appropriate rate to provide a perception of movement to the user 18. Thus, the user 18 may be provided with additional information regarding a position of the instrument 62, such as being in contact with of the RV septum 316.

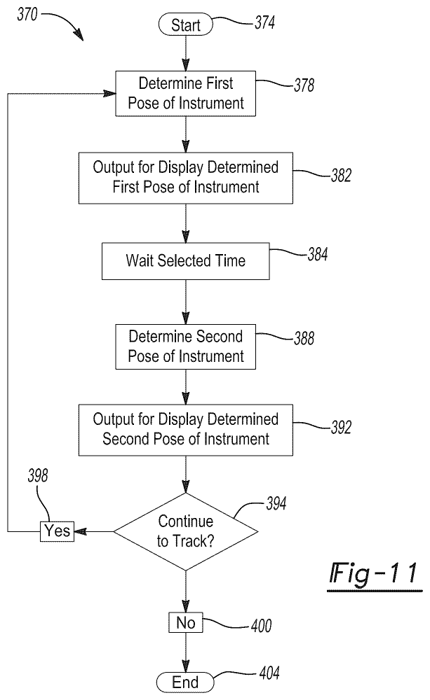

With reference to FIG. 11, a method or process 370 is illustrated. The process 370 may be a process that is executed by one or more of the processing units, such as processing unit 74. The method 370 may begin at start Block 374. After starting the process in Block 374, the process 370 may determine a first position or pose of the instrument in Block 378. Determining the first pose of the instrument may be performed with the navigation system, as discussed above. The pose of the instrument 62 may be known relative to the patient 14, or portions thereof such as the heart 15, in light of the tracking of the patient with the patient tracker 58.

After determining a pose of the instrument at a first time, an output for display of the determined pose of the instrument may be made in Block 382. The output of the determined pose may be based upon the registration of the image 78 and/or the avatar 120 to the physical space defined by the patient 14, as discussed above. Thus, the output may display the graphic representation 90 at a first position, for example of the first pose 90t1, as illustrated in FIG. 10A.

After determining and outputting the first pose, a wait or pause for a selected time may occur in Block 384. The wait for a selected time may be any appropriate time, such as about one second to about 120 seconds. The wait time may be any appropriate time to assist in illustrating a motion to the user 18, as discussed above. After the wait time, the second pose may be determined in Block 388. The second pose may be determined in the same manner as the first pose was determined in Block 384. An output for display of the second pose may be made in Block 392 to display the graphical representation of the instrument 62 at a second time, such as the graphic representation 90t2. As noted above, therefore, the pose of the instrument 62 may be displayed over a series of the events and/or time to show movement to the user 18.

A determination can be made to continue tracking at Block 394. If tracking is to be continued, a YES path 398 may be followed to again determine a first or subsequent pose in Block 378. The process 370 may provide an illustration to the user 18 for an appropriate period of time. If a tracking is not to be continued, a NO path 400 may be followed to end the process in Block 404. The process 370 may be used to illustrate and determine a pose of the instrument 62 over time for illustration relative to the image 78, as illustrated in FIGS. 10A and 10B.

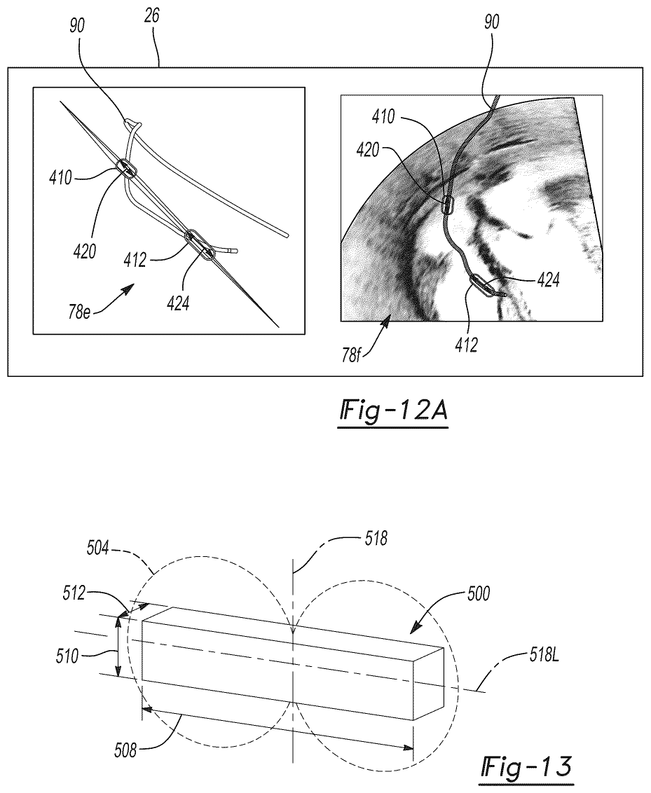

The display 26 may display the image of the patient 14 that is acquired with the imaging system 12. As discussed above, the image 78 may be an image that is acquired in a plane from the ultrasound housing 16. Therefore, rotation of the image may change a perspective of the image 78. With reference to FIG. 12A, the image may be viewed over the plane 78f or from an edge 78e. The edge image 78e may allow for a viewing and understanding of a perspective of the instrument 62, as illustrated by the graphic representation 90, relative to a plane of the image. In various embodiments, the image acquired with the imaging system 12 may be a static image that is acquired with the imaging system 12. It is understood, however, that a live or current image may also be acquired and displayed. Nevertheless, the position of the instrument 62 relative to the plane of the image may be illustrated by rotating the image, such as showing it from an edge as illustrated in the edge image 78e.

Further, the distance of the instrument 62 that is within the plane of the image may also be illustrated. For example, a first and second distance icon 410, 412 may be included or illustrated superimposed on both of the images 78e, 78f. The distance icons may be used to illustrate the distance that the instrument 62 is in the plane of the image. Thus, the user 18 may also understand that the portion of the instrument that is within the plane of the image and that other portions are not within the plane of the image.

The distance icon 410, 412 may be sized depending upon the length or portion of the instrument 62 that is within the plane of the image 78. Therefore, the first distance icon 410 may have a first distance or size 420. The second distance icon 412 may have a second distance or size 424. The two dimensions 420, 424 may be different or the same depending upon the amount of the instrument or length of the instrument that is within the plane of the image 78. If the instrument has a greater length within the plane, the distance icons 410, 412 may have greater dimensions. If any dimension of the instrument is smaller in the plane, then the dimension icons may be smaller. Regardless, the dimension icons 410, 412 may be displayed, such as superimposed on, the image 78 to represent the distance of the instrument within the image plane. This may allow the user 18 to understand or determine the distance of the instrument 62 within a plane of the imaging system and/or assist in moving of the imaging system to acquire a different perspective than in the image 78 relative to the instrument 62.

According to various embodiments, the display 26 may display the image of the patient 14 that is acquired with the imaging system 12 at more than one location. In other words, more than one image may be displayed for each position of the imaging system 12 at which image data is acquired. Moreover, one or more image data portions may be merged and/or used in a reconstruction of a displayed image. As discussed above and herein, all of the images and/or the reconstructed image may be registered to the patient 14 due at least to the tracking of the imaging system 12.

With reference to FIG. 12B, for example, the image 78 may be an image that is acquired in a plane from the ultrasound housing 16. Therefore, rotation of the image may change a perspective of the image 78. The image may be viewed as acquired at a first location (e.g., inferior from the suprasternal notch) as over a plane 78f′ or from an edge 78e′. These two views may be able to provide information to the user, as discussed above including location and/or dimension that the instrument graphic representations 90 passes through the plane 410′, 412′, 420′, 424′. Further, the image may be viewed as acquired at a second location (e.g., between selected ribs) as over a plane 78f″ or from an edge 78e″. These two views may be able to also provide information to the user, as discussed above including location and/or dimension that the instrument graphic representations 90 passes through the plane 410″, 412″, 420″, 424″. Given that the image portions 78e′, 78f′ are acquired at a different position than the image portions 78e″, 78f″ the graphic representations 90 is illustrated to pass through the planes at the different positions and/or dimensions 410′, 412′, 420′, 424′, 410″, 412″, 420″, 424″ and this information is displayed for the user. Further, it is understood that any appropriate number of location images may be acquired and/or displayed and two is merely exemplary.

Again, the edge image may allow for a viewing and understanding of a perspective of the instrument 62, as illustrated by the graphic representation 90, relative to a plane of the image. In various embodiments, the image acquired with the imaging system 12 may be a static image that is acquired with the imaging system 12. It is understood, however, that a live or current image may also be acquired and displayed. Nevertheless, the position of the instrument 62 relative to the plane of the image may be illustrated by rotating the image, such as showing it from an edge as illustrated in the edge image.

The instrument 62 may be tracked with any appropriate tracking system. As noted above, tracking and/or navigation systems include those disclosed above and those disclosed in U.S. Pat. No. 7,697,972 issued Apr. 13, 2010; U.S. Pat. No. 8,046,052 issued Oct. 25, 2011; U.S. Pat. No. 8,180,428 issued May 15, 2012; and U.S. Pat. No. 8,663,120 issued Mar. 4, 2014, all of which are herein incorporated by reference. The navigation system may allow for a determination of a pose of the instrument 62 in a physical location and/or orientation of the instrument 62 in the physical space. The tracked pose may include a location which includes X, Y, and Z axis positions and/or orientation which may include yaw, pitch, and roll orientations in space. A tracking and navigation system may also include one or more permanent magnets and/or magnetometers.

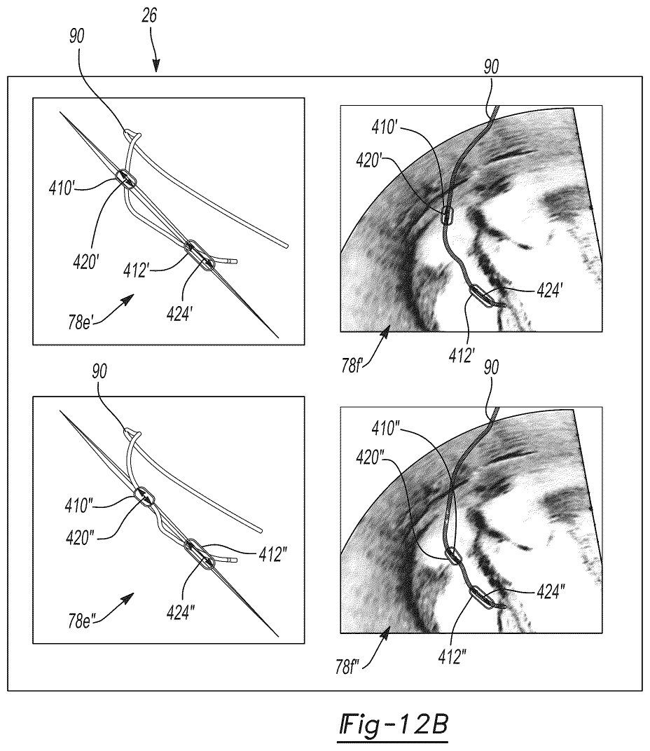

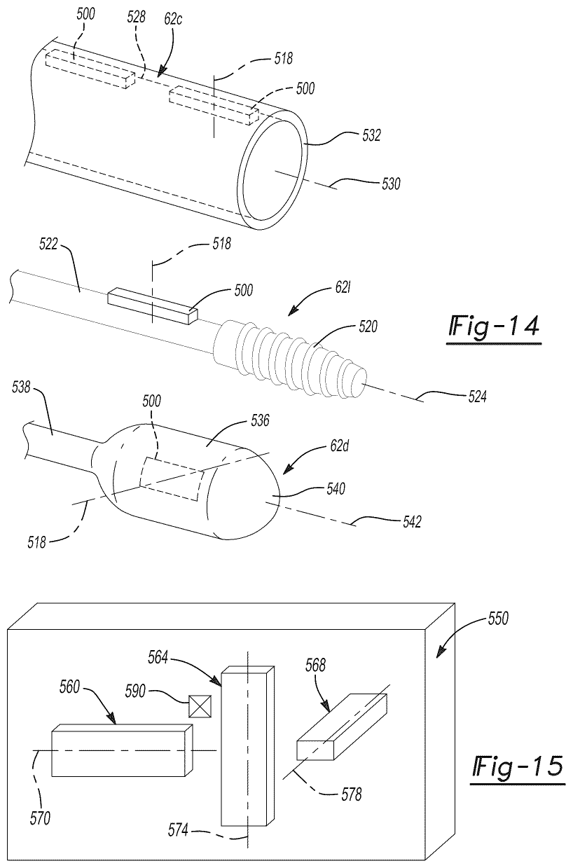

With reference to FIGS. 13, 14, 15, and 16, a magnet 500 may be positioned with, in or on the instrument 62 as the instrument tracking device 60, according to various embodiments. As an example, the magnet 500 may be fixed relative to the instrument, which can comprise at least one of a catheter 62c, a dilator 62d, or other portion or device that may be temporarily placed in and then removed from the patient 14. The magnet 500 may also be placed on a lead 621, or a leadless pacemaker or a delivery system therefor (not shown) that may be inserted or implanted in the patient 14. Regardless, as discussed herein, the magnet 500 may emit a magnetic field 504 relative thereto that may be detected and used to track the instrument 62.

With initial reference to FIG. 13, the magnet 500 may be a magnet that has a selected dimension 508 as a first dimension, a second dimension 510, and a third dimension 512. The three dimensions 508, 510, and 512 may allow for generation of the magnetic field 504 to define an axis 518 relative to the magnet 500. Therefore, the axis 518 may be determined relative to the magnet 500 when the field 504 is sensed, as discussed herein. It is understood that the magnet 500 may be magnetized in any appropriate orientation, thus the magnet 500 may also define an axis 5181 that is a long axis of the magnet 500 if the magnet is magnetized along its long axis.

The magnet 500 may be formed of a selected material that enables the magnet to generate a magnetic field on its own, without need for any external power. Thus, the magnet 500, in various embodiments, forms the magnetic field alone. In other words, the magnet 500 may be the only member formed the magnetic field according to various embodiments. The magnet 500 may be formed of selected materials, including more than one material, such as a rare earth materials. Rare earth material magnets include those such as neodymium, iron, and boron magnets The field 504 of the magnet 500 may be an appropriate strength, such as about 0.001 Tesla (T) to about 5 Tesla, including about 0.1 T to about 3T. Detectability of the field could be around 1 nT. According to various embodiments, magnetic tagging may be used such as disclosed in U.S. Pat. No. 6,329,916.

The field 504 of the magnet 500 may be sensed exterior to the patient 14, as discussed further herein, when the magnet 500 is within the patient 14. The magnet 500 may include a dimension or size that allows it to be inserted into the patient 14, such as into and relative to the heart 15 of the patient 14. Thus, the magnet 500 may be positioned relative to the heart 15 and be sensed exterior to the patient 14, as discussed herein.

The magnet 500, as noted above, may be positioned on, in or relative to one or more instruments 62 that is positioned within the patient 14. The magnet 500 may be used to determine a pose of the instrument 62. The pose may include up to six degrees of freedom that may include x, y, and z axis and/or orientation including yaw, pitch, and roll.

For example, during positioning of a cardiac device, including a cardiac defibrillator, cardiac pacemaker, or the like, the lead 621 may be positioned within that the patient 14 and implanted into the heart 15. In various embodiments, the lead 621 may be permanently positioned, such as within the patient 14 during the procedure and retained in the patient 14 after the procedure. The lead 621 may include various portions including a fixation member 520 and a conductor 522. The magnet 500 may be fixed relative to the lead 621. The magnet 500 may include the axis 518 that is at a known orientation relative to various portions of the lead 621 including a long axis 524 of the lead 621 and a selected distance from the fixation member 520. Thus, the pose of the lead 621 may be determined based upon sensing the magnetic field 504, determining the axis 518 and the known or predetermined location of the magnet 500 relative to other portions of the lead 621.

The magnet 500 may also be positioned on, in or relative to other instruments, such as the catheter 62c. The catheter 62c may include an interior cannula 528 through which various members may be passed, such as the lead 621 or other instruments. They magnet 500, however, may be positioned within the catheter 62c such as in a wall thereof. The magnet 500 may define the field 504 that defines the axis 518. Again, the orientation of the axis 518 may be known relative to a portion of the catheter 62c, such as a long axis of the catheter 530. Further, a distance of the axis 518 relative to a distal end 532 of the catheter 62c may also be known. Thus, a pose of the catheter 62c may be determined based upon sensing the field of the magnet 500 and determining the location and/or orientation of the axis 518. The magnet 500 can also be shaped with a hole along an axis of the magnet (e.g., the long axis 518L) to allow passage of a guide wire, lead, etc. assuming a cylindrical shape for the magnet and coaxial placement of it within the catheter.

As a further example, the dilator 62d may also incorporate the magnet 500. The dilator 62d may include a distal end 536 that is enlarged relative to an elongated portion or shaft 538. The end 536, therefore, may allow for inclusion of the magnet 500 therein. The magnet 500 may include a selected volume that may be maintained within the distal end 536. Thus, the magnet 500 may generate the field that defines the axis 518. The location and/or orientation of the axis 518 may again be known relative to a distal tip 540 of the dilator 62d and also relative to a long axis 542 of the dilator 62d. This allows a pose of the dilator 62d, particularly the end 536 thereof, to be determined with the navigation system when sensing the field 504 of the magnet 500.

Thus, the magnet 500 may be associated with, such as fixed to or incorporated into, one or more of the instruments 62. The instrument 62 may be positioned within the patient 14 and the magnetic field 504 of the magnet 500 may be sensed. The magnetic field 504 may be sensed with an appropriate instrument, such as a magnetometer. The field sensed with the magnetometer may then be used to determine a pose of the magnet 500 and, therefore, a pose of the related instrument, such as that described above.

Moreover, while each of the instruments is illustrated and/or described with at least one magnet 500, each instrument may have more than one magnet 500. For example, the catheter 62c may have a plurality of magnets spaced along a length of the catheter 62c. Thus, a pose of each of the magnets 500 along the length and, therefore, each portion including one of the magnets may be determined. This may allow a pose of more than one portion of the instrument to be determined simultaneously. According to various embodiments, only one magnet 500 may be provided or a plurality of the magnets 500 may be provided on an instrument and/or relative to the patient 14.

The magnet 500 may be understood to be a magnetic field generator that may generate a magnetic field as a permanent magnet. As discussed above, a permanent magnet includes material that generates the magnetic field 504 alone with no external force or power. The permanent magnet and/or field generated thereby, therefore, may differ from a magnetic field that is generated with a coil through which a direct current (DC) or alternating current (AC) is passed. As understood by one skilled in the art, a direct or alternating current may be passed through a coil of wire to generate a magnetic field. The coil must be connected to a power source to generate and drive the current through the wire and to form a field. Only the permanent magnet, however, generates the magnetic field with no current being supplied thereto. The magnetic field of the permanent magnet may be sensed by a magnetic field sensing device, such as a hall effect sensor, and an Anisotropic Magneto Resistive sensor (AMR), or other appropriate DC magnetic field sensor. Low-pass filter to limit the bandwidth and enhance signal-to-noise ratio (SNR). Filter bandwidth needs to be appropriately selected to provide desired response time while maintain the highest SNR. Further, AMR sensors may include Analog Devices Anisotropic Magnetoresistive sensors, which are targeted for typical EM localization applications. The AMR sensors also sense DC magnetic fields. The six degree of freedom (6DOF) configuration may be affixed to the skin at one or more, including three or more, discrete locations should be able to track a position of the magnet 500 in the patient 14. In various embodiments, a fiducial marker may be incorporated with the sensor so each could precisely localized with a tracking system (e.g., an infrared (IR) optical tracking system) and a point probe. IR reflective spheres could be used for real-time direct tracking of the sensors rather than periodically touching off on the fiducial markers with a probe. As an example, a network of such sensors may work similar to a cell tower system used for continuous telecommunications coverage.

A magnetic sensor may include those as noted above. A magnetic sensor may be oriented relative to a fixed position and sense a field along at least one axis. As illustrated in FIG. 15, a magnetic sensing assembly 550 may include a selected number of magnetic sensors. In various embodiments, the magnetic sensing assembly may include three magnetic sensors including a first magnetic sensor 560, a second magnetic sensor 564, and a third magnetic sensor 568. It is understood, however, that the magnetic sensing assembly 550 may include more or fewer than three magnetic sensors. For example, the assembly 550 may include four or more sensors or two or fewer. More sensors may allow for greater resolution of location and orientation, if selected.

In various embodiments, three magnetic sensors 560-568 may be oriented within the magnetic sensing assembly 550 to sense a magnetic field along one of three respective axes. The first magnetic sensor 560 may sense the field along a first axis 570, the second magnetic sensor may sense the field along a second axis 574, and the third sensor 568 may sense a magnetic field along a third axis 578. Each of the three axes 570-578 may be oriented substantially orthogonal to one another. Therefore, the three-dimensional position of a sensed magnetic field may be determined relative to a point defined relative to the magnetic sensing assembly 550. For example, the three-dimensional position may be determined relative to an origin point 590 established relative to the magnetic field sensing assembly 550.

The pose (including a position and/or orientation) of the axis 518 of the magnet 500 may be known relative to an orientation of the magnet 500 and also relative to a configuration of the instrument 62. An orientation of the magnet 500 may be determined and a related orientation of the instrument 62 may also be determined relative to the origin point 590. As the orientation and/or location of the magnet 500 may be determined, the pose of the magnet 500 may be determined and the associated pose of the instrument 62 may also be determined. Thus, the magnetic sensing assembly 550 may sense the magnetic field of the magnet 500 and a determination of the pose of the magnet and the related instrument may be made based thereon.

Without being bound by the theory, each of the three magnetic sensors 560, 564, 568 may sense the magnetic field independently when the magnet 500 is in any specific position relative thereto. Therefore, a determination may be made, based upon the sensed field, of the location and orientation of the magnet 500 relative to the magnetic sensing assembly 550 including the origin point 590. The sensed field may then be used by the navigation system to determine the pose of the instrument 62 associated with the magnet 500.

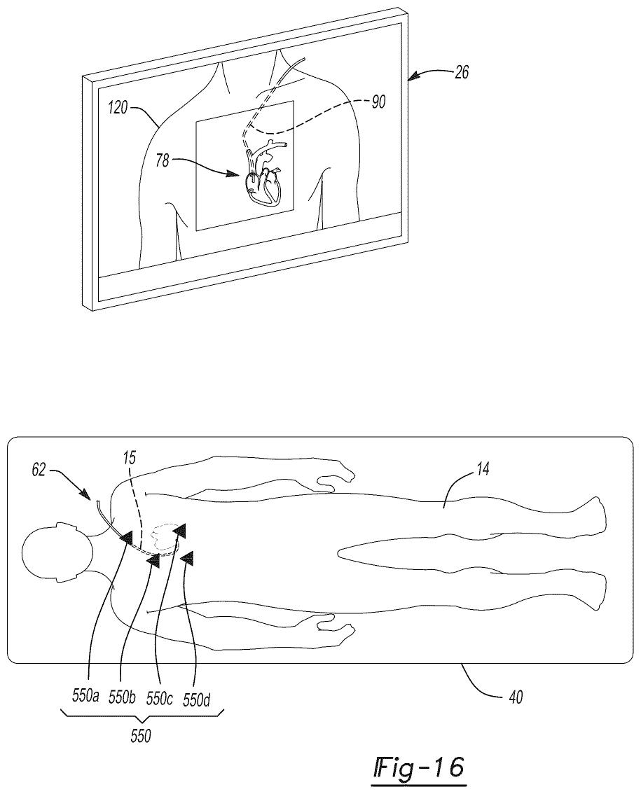

The magnetic sensing assembly 550 may be positioned on or near the patient 14 at any appropriate location. With reference to FIG. 16, the magnetic sensing assembly 550 may be provided on the patient 14 at a single location or at a plurality of locations. In various embodiments, for example, the magnetic sensing assembly 550 may be positioned on the patient 14 at four positions or locations relative to the heart 15 of the patient 14. As illustrated in FIG. 16, four different magnetic sensing assemblies may be positioned, referenced as 550a, 550b, 550c, and 550d. Each of the magnetic sensing assemblies 550 may be positioned at different locations relative to the heart 15. Therefore, as the instrument 62 moves relative to the heart, the magnet 500 may be sensed by one or more of the magnetic sensing assembl(ies) 550 during the procedure.

While the magnet 500 may be sensed with the magnetic sensing assembl(ies) 550, the navigation system may determine the pose of the instrument and display it with the graphic representation 90 on the display device 26. As discussed above, the graphic representation 90 may be displayed relative to the avatar 120 and/or the image 78. Thus, the user 18 may understand the pose of the instrument 62 during the procedure. In various embodiments, the pose of the instrument 62 may be determined based upon a sensed magnetic field sensed by any one of the magnetic assemblies 550 and/or two or more of the magnetic assemblies 550. The navigation system may determine the pose of the instrument 62 based upon the received one or more signals. As discussed herein, the pose of the magnet 500 may be determined and a pose of the instrument 62 may be determined based thereon. Thus, the pose of the instrument 62 may be displayed with a graphical representation on the display device 26, including the superimposed graphic representation 90.

In various embodiments, for example, the pose of the instrument 62 may be determined with the first magnetic sensing assembly 550a when the magnetic sensing assembly 550a is nearest the magnet 500. As the instrument 62 moves past the magnetic sensing assembly 550a and closer to the magnetic sensing assembly 550b, the magnetic sensing assembly 550b may be used to determine the pose of the instrument 62. The choice of the specific magnetic sensing assembly, if more than one is provided, used for the determination of the pose of the instrument 62 at a particular moment in time may be based upon the strength of the field sensed from the magnet 500 associated with the instrument 62. Thus, the navigation system may determine the magnetic sensing assembly that sensed the strongest magnetic field and make the determination based upon that magnetic sensing assembly signal. It is understood, however, that the pose of the instrument 62 may be determined at any particular time based upon the signal received from more than one of the magnetic sensing assemblies 550.

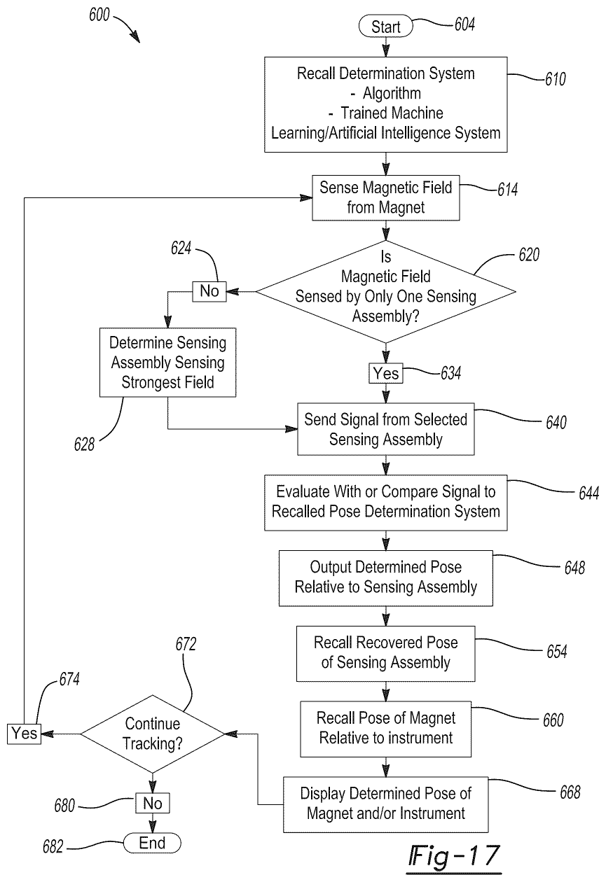

With continuing reference to FIGS. 13-16, and additional reference to FIG. 17, a process 600 for displaying the pose of the instrument 62 is disclosed. The process 600 may be included in an algorithm and/or system having instructions that are executed with the processing unit, such as the processing unit 74. Therefore, it is understood that the process 600 may be substantially an automatic process. Nevertheless, the process 600 may include various manual inputs, such as those from the user 18.

The process 600 may begin at start Block 604. Starting in Block 604 may include initializing the system, preparing the patient 14, or otherwise associating the instrument 62 in a navigation space relative to the patient 14. At the start of the procedure in Block 604, according to various embodiments, a zeroing step may be made to account for the effects of earth's magnetic field. During this step, the magnet 500 should be away from the sensors and measured output due to earth's magnetic field should be subtracted to make all sensor outputs zero.

After starting the process in Block 604, a recall of a pose determination system may be made in Block 610. The pose determination system may be any appropriate pose determination system. For example, an algorithm may determine a pose of the instrument relative to one or more of the magnetic sensing assemblies 550. The algorithm, therefore, may be recalled or loaded into memory for access by the processing unit during execution of the method 600. Additionally, a machine learning system, including an artificial intelligence or neural network, may also be trained and recalled. The machine learning system may have been trained based upon moving in the magnet relative to one or more of the magnetic sensing assemblies 550 to train a machine learning system. The machine learning system may then be used to determine a pose of the magnet 500 during use of the magnet 500 in a procedure on the patient 14. Thus, the machine learning system may be trained in any appropriate time, e.g., any time prior to the instant procedure, to determine a pose of the magnet relative to one or more of the sensing assemblies 550. The trained system may also be updated with information from one or more procedures to assist in enhancing a validity of the trained system.

A field from a magnet, for example a DC magnetic field, may be sensed in Block 614. As discussed above, a permanent magnet may have a DC magnetic field and may be sensed by any appropriate sensing system, including the magnetic sensing assembly 550. Therefore, once the procedure 600 starts in Block 604, a field may be sensed in Block 614. As noted above more than one magnetic sensing assembly 550 may be provided and employed during a procedure. Thus, the magnetic field may be sensed by more than one magnetic sensing assembly 550. The process 600 allows for determination of whether the magnetic field is sensed by more than one sensing assembly 550 in Block 620. The determination may be made that the magnetic field is sensed by only one magnetic sensing assembly, by a plurality of magnetic sensing assemblies, or by no magnetic sensing assemblies. In other words, the process 600 may allow for a determination of the magnetic field is sensed by one or more of the magnetic sensing assemblies.

If a determination is made that the field is not being sensed by only one the magnetic sensing assemblies in Block 620, a NO path 624 may be followed. The NO path 624 leads to a determination of which sensing assembly senses the strongest field in Block 628. The determination of which sensing assembly senses the strongest field may allow for a choice of which sensing assembly to utilize to determine a pose of the magnet(s). Thus, the determination of the pose of the magnet(s) may be based upon the strongest sensed field even when there is a plurality of the sensing assemblies 550. Further, in various embodiments, signals may not be discarded even though weaker than the best signal. It can be used to improve position and orientation registration.

If the determination is made that only a single one of the sensing assemblies 550 is sensing the field, a YES path 634 may be followed. A signal, after following the YES path 634, may be sent from the selected sensing assembly in Block 640. The signal from the sensing assembly may include a signal that is an aggregate of all of the three orthogonal axes of all the sensing portions 560, 564, and 578 or an individual signal from each of the sensing portions. The signal, therefore, may be based upon the sensed field from the magnet 500.

After sending the signal in block 640, the signal may be evaluated according to the recalled determination system in Block 644. As discussed above, the determination system may be any appropriate determination system including a specific algorithm, a trained machine learning system (e.g., a neural network), or other appropriate determination system. The determination system may be included in instructions that are executed by the processing unit 74. The pose may, therefore, be determined based upon the signal sensed from the selected sensing assembling from Block 640. The evaluation allows for an output of the determined pose relative to the sensing assembly in Block 648.

The pose relative to the sensing assembly 550 allows for a determination of the pose of the magnet 500 relative to the sensing assembling 550 from which the signal is sensed based upon sensing the magnetic field. The pose relative to the sensing assembly 550 may be used to determine the pose of the magnet relative to other portions, such as the image 78 and/or the avatar 120.

To facilitate generation of the graphic representation 90 for display relative to the registered image, such as the image 78 and/or the avatar 120, a recall of the registered pose of the sensing assembly may be made in Block 654. The pose of the sensing assembl(ies) 550 may be determined by tracking the sensing assembl(ies) 550 relative to the patient tracker 56, the localizer(s) 50, 55, or other appropriate mechanisms. For example, the sensing assembl(ies) may be positioned at known or predetermined positions relative to the patient 14 including the heart 15. As is illustrated in FIG. 16, the sensing assembl(ies) 550 may be positioned at a specific geometric position relative to the heart 15 of the patient 14. Thus, the sensing assemblies 550 may have known poses relative to the heart 15. An image of the heart, such as the image 78, may then have a known pose relative to the sensing assemblies 550. A signal sensed by the sensing assemblies 550 from the magnets associated with the instrument 62 may then be known relative to the sensing assemblies 550 for determining a pose to generate the graphic representation 90.

The system may further recall a pose of the magnet relative to the instrument in Block 660. As discussed above, the position of the magnet 500 relative to the instrument 62 is known. Illustrated in FIG. 13, the axis 518 may be defined relative to the magnetic field 504. It is understood, however, that the longitudinal axis 5181 may also be determined relative to the magnet 500. In various embodiments, for example, the magnetic field 504 may be rotated substantially 90° relative to the magnet as illustrated in FIG. 13 and allow the axis 5181 to be the axis defined by the magnetic field 504. Regardless, the pose of the magnet relative to a portion of the instrument, such as the respective long axes of the respective instruments 524, 530, 542, may be recalled in Block 616. After recalling the pose of the magnet relative to the instrument in Block 560, a determination of the pose of the instrument may be made in Block 664 and may be displayed in Block 668.

Display of the determined pose may be made as discussed and illustrated above, such as FIG. 16. The graphic representation 90 of the instrument 62 may be displayed as the pose of the instrument based upon the determined pose of the magnet relative to the magnetic sensing assemblies 550. The user 18 may therefore view the display device 26 and understand the pose of the instrument 62 relative to portions of the patient 14 that are not visible, such as the heart within patient 14.

After displaying the pose in Block 668, a determination of whether tracking is to be continued may be made in Block 672. If tracking is to continue, a YES path 674 may be followed to sense a magnetic field from the magnet in Block 614. Thus, the process 600 may loop to provide a real time sensing and display of the instrument 62 relative to a selected portion, such as the image 78, with a display device 26. If tracking is not to be continued, a NO path 680 may be followed to end the process in Block 682. Thus, the process may end when tracking is complete, such as may be selected by the user 18. The process 600 may allow for a tracking and display of a pose of the instrument 62 over time, based on the position of the magnet 500 relative to the instrument 62 as it is moved relative to the tracking magnetic sensing assembl(ies) 550. The magnetic sensing assembl(ies) 550 and the magnet 500 allow the pose of the instrument 62 to be tracked and determined without requiring an external energy emitting system. Rather, the permanent magnet generates the magnetic field that is sensed by the magnetic sensing assembl(ies) to determine the pose of the magnet 500. Thus, a field may not be generated by the localizer to define the navigation space but rather the magnetic field from the magnet 500 in the patient 14 may be sensed to make the pose determination.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit that may also be referred to as a processor. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

The following examples are a non-limiting list of clauses in accordance with one or more techniques of this disclosure.

Example 1. A system to track a device relative to a heart of a subject, comprising: an ultrasound imaging system configured to acquire an image data of the heart of the subject; a tracking system; a tracking device configured to be tracked by the tracking system; an instrument configured to be moved relative to the heart of the subject, wherein the tracking device is associated with the instrument; a processing unit configured to execute instructions to: register the image data to the subject; generate an image of the heart of the subject; and generate a graphical representation of the tracked pose of the instrument relative to the generated image.

Example 2. The system of Example 1, wherein ultrasound imaging system is configured to generate image data of the subject in a plane relative to an ultrasound transducer.

Example 3. The system of Example 2, wherein the generated graphical representation includes (i) a representation of the instrument and (ii) a representation of a dimension that the instrument intersects the plane of the image data acquired with the imaging system.

Example 4. The system of Example 2 or Example 3, further comprising: an imaging system tracking device; wherein a pose of the plane is determined by tracking the ultrasound imaging system, wherein the imaging system tracking device is associated with the ultrasound transducer.

Example 5: The system of any of Examples 1-3, wherein the processing unit is configured to execute further instructions to: recall an atlas of a heart operable to be related to the heart of the subject; relate the atlas to the generated image of the heart; and determine a location of at least one portion of the heart in the generated image data based on the recalled atlas of the heart.

Example 6. The system of Example 5, wherein the atlas is registered to the generated image of the heart at least by an identification of a landmark in both the atlas and the generated image of the heart.

Example 7. The system of Example 6, wherein the image data includes ultrasound image data in at least a first plane and a second plane.

Example 8. The system of any of Examples 1-3, wherein the instrument includes at least one of a catheter or a lead.

Example 9. The system of Example 8, wherein the catheter includes a cannula through which the lead is configured to pass to the heart of the subject.

Example 10. The system of any of Examples 1-3, wherein the tracking system is configured to track the pose of the tracking device over time; wherein the generated graphical representation of the tracked pose of the instrument relative to the generated image changes over time based on the tracked pose of the tracking device over time.

Example 11. A method to track a device relative to a heart of a subject, the method comprising: acquiring an ultrasound image data of the heart of the subject from an ultrasound imaging system; tracking a pose of a tracking device with a tracking system; providing an instrument configured to be moved relative to the heart of the subject, wherein the tracking device is associated with the instrument; executing instructions with a processing unit to: register the image data to the subject; generate an image of the heart of the subject; and generate a graphical representation of the tracked pose of the instrument relative to the generated image.

Example 12. The method of Example 11, wherein acquiring the ultrasound image data includes acquiring ultrasound image data in at least a plane relative to an ultrasound transducer.

Example 13. The method of Example 12, wherein executing instructions with the processing unit to generate the graphical representation includes generating: (i) a representation of the instrument, and (ii) a representation of a dimension that the instrument intersects the plane of the image data acquired with the ultrasound imaging system.

Example 14. The method of Example 13, further comprising: tracking an imaging system tracking device; determining a pose of the plane by tracking the imaging system tracking device associated with the ultrasound transducer of the ultrasound imaging system.

Example 15. The method of Example 11, wherein executing instructions with the processing unit includes executing further instructions to: recall an atlas of a heart operable to be related to the heart of the subject; relate the atlas to the generated image of the heart; and determine a location of at least one portion of the heart in the generated image data based on the recalled atlas of the heart.

Example 16. The method of Example 11, further comprising: registering the atlas to the generated image of the heart at least by an identification of a landmark in both the atlas and the generated image of the heart.

Example 17. The method of Example 16, wherein acquired the image data includes acquiring ultrasound image data in at least a first plane and a second plane.

Example 18. The method of Example 11, further comprising: determining a pose of at least one of a catheter or a lead by tracking the tracking device.

Example 19. The method of Example 18, further comprising: providing the catheter to include a cannula through which the lead is configured to pass to the heart of the subject.

Example 20. The method of Example 11, further comprising: tracking the pose of the tracking device over time; executing the instructions with the processing unit to generate the graphical representation of the tracked pose of the instrument relative to the generated image to change over time based on the tracked pose of the tracking device over time.

Example 21. A system to assist a user in navigating an instrument relative to a subject, comprising: a display device configured to be viewed by the user; a tracking system configured to track an instrument and determine a pose of the instrument relative to the subject; a processor configured to execute instructions to: receive information regarding the subject; recall a standard avatar; generate a subject specific avatar at least by morphing the recalled standard avatar based on the received information regarding the subject; output the generated subject specific avatar; wherein the display device is configured to display the generated subject specific avatar and a graphical representation of the instrument superimposed on the generated subject specific avatar based on the tracked pose of the instrument.

Example 22. The system of Example 21, further comprising: the instrument; and a tracking device associated with the instrument that is tracked with the tracking system.

Example 23. The system of Example 22, wherein the instrument comprises an elongate endovascular device.

Example 24. The system of Example 22, wherein the instrument comprises an intracardiac device.

Example 25. The system of Example 22, wherein the instrument comprises a guidewire.

Example 26. The system of Example 22, wherein the instrument comprises a catheter.

Example 27. The system of Example 22, wherein the instrument comprises a delivery system for an implant.

Example 28. The system of Example 22, wherein the instrument comprises a tunneling tool.

Example 29. The system of Example 22, wherein the instrument comprises an energy delivery device.

Example 30. The system of Example 22, wherein the instrument comprises an electrode configured to stimulate a heart of the subject.

Example 31. The system of any of Examples 21-30, further comprising: a subject tracker, wherein the subject tracker is fixed relative to the subject to track a pose of the subject.

Example 32. The system of Example 31, wherein the subject tracker is operable to be tracked with the tracking system and the processor is configured to execute further instructions to maintain a registration of the generated subject specific avatar with the subject.

Example 33. The system of Example 31, wherein the received information regarding the subject includes subject specific information.

Example 34. The system of Example 33, wherein the received information regarding the subject includes tracked poses of at least two points of the subject.

Example 35. The system of any of Examples 21-30, wherein the tracking system includes a localizer; wherein the subject is positioned relative to the localizer at a predetermined position; wherein the predetermined position is operable to allow registration of the subject to the output generated subject specific avatar.

Example 36. A method to assist a user in navigating an instrument relative to a subject by selecting and sizing an avatar of a subject, the method comprising: executing instructions with a processor to: receive information regarding the subject; recall a standard avatar; generate a subject specific avatar at least by morphing the recalled standard avatar based on the received information regarding the subject; and output the generated subject specific avatar; and displaying the generated subject specific avatar including relative positions of portions of the subject based on the received information regarding the subject.

Example 37. The method of Example 36, further comprising: tracking an instrument and determining a pose of the instrument relative to the subject; and displaying a graphical representation of the instrument superimposed on the displayed subject specific avatar based on the tracked pose of the instrument.

Example 38. The method of Example 36, wherein generating the subject specific avatar at least by morphing the recalled standard avatar based on the received information regarding the subject includes displaying the subject specific avatar to have subject portions based on relative positions of the subject portions in the subject.

Example 39. The method of Example 38, further comprising: providing a tracking device associated with the instrument that is tracked with the tracking system.

Example 40. The method of Example 39, wherein the instrument comprises an elongate endovascular device.

Example 41. The method of Example 39, wherein the instrument comprises an intracardiac device.

Example 42. The method of Example 39, wherein the instrument comprises a guidewire.

Example 43. The method of Example 39, wherein the instrument comprises a catheter.

Example 44. The method of Example 39, wherein the instrument comprises a delivery system for an implant.

Example 45. The method of Example 39, wherein the instrument comprises a tunneling tool.

Example 46. The method of Example 39, wherein the instrument comprises an energy delivery device.

Example 47. The method of Example 39, wherein the instrument comprises an electrode configured to stimulate a heart of the subject.

Example 48. The method of Example 39, further comprising: providing a subject tracker to be fixed relative to the subject to track a pose of the subject.

Example 49. The method of Example 48, wherein the subject tracker is operable to be tracked with the tracking system and the processor is configured to execute further instructions to maintain a registration of the generated subject specific avatar with the subject.

Example 50. The method of Example 36, wherein the received information regarding the subject includes subject specific information.

Example 51. The method of Example 50, wherein the subject specific information includes tracked poses of at least two points of the subject.

Example 52. The method of Example 36, further comprising: providing a tracking system that includes a localizer; providing the localizer to be positioned at a predetermined position relative to the subject; registering the subject to the generated subject specific avatar based on the predetermined position relative to the subject of the localizer.

Example 53. A method to assist a user in navigating an instrument relative to a subject by selecting and sizing an avatar of a subject, the method comprising: executing instructions with a processor to: receive information regarding the subject; recall a standard avatar; generate a subject specific avatar at least by morphing the recalled standard avatar based on the received information regarding the subject; and output the generated subject specific avatar; displaying the generated subject specific avatar including relative positions of portions of the subject based on the received information regarding the subject; tracking an instrument and determining a pose of the instrument relative to the subject; registering the displayed generated subject specific avatar to the subject; and displaying a graphical representation of the instrument superimposed on the displayed subject specific avatar based on the tracked pose of the instrument.

Example 54. The method of Example 53, wherein registering the displayed generated subject specific avatar to the subject comprises: providing a localizer at a predetermined position relative to the subject; and registering the subject to the generated subject specific avatar based on the predetermined position relative to the subject of the localizer.

Example 55. A system operable to determine a pose of an instrument, comprising: a permanent magnet configured to generate a magnetic field that defines a magnet axis, wherein the permanent magnet is sized to be incorporated with the instrument; a magnetic field sensing assembly comprising, a first magnetic field sensor configured to sense the magnetic field relative to a first axis relative to the magnetic field sensing assembly, a second magnetic field sensor configured to sense the magnetic field relative to a second axis relative to the magnetic field sensing assembly, and a third magnetic field sensor configured to sense the magnetic field relative to a third axis relative to the magnetic field sensing assembly, wherein the first axis, the second axis, and the third axis are orthogonal to each other; and a processing unit configured to receive a signal from the magnetic field sensor assembly regarding the sensed magnetic field relative to each of the first axis, the second axis, and the third axis and execute instructions to determine the pose of the instrument.

Example 56. The system of Example 55, further comprising: the instrument; wherein the instrument is configured to be inserted within at least a portion of a subject.

Example 57. The system of Example 56, wherein the subject includes a human subject.

Example 58. The system of Example 57, wherein the portion of the subject includes a vessel of the human subject.

Example 59. The system of any of Examples 55-58, wherein the permanent magnet includes a plurality of permanent magnets; wherein each permanent magnet of the plurality of permanent magnets is spaced apart from the any other permanent magnet of the plurality of the permanent magnets along the instrument.

Example 60. The system of any of Examples 55-58, wherein the permanent magnet includes a single permanent magnet having a magnetic field that is sensed to determine the pose of a selected portion of the instrument.

Example 61. The system of any of Examples 55-58, wherein the magnetic field sensing assembly includes a plurality of magnetic field sensing assemblies.

Example 62. The system of Example 61, wherein the processing unit is configured to execute instructions to determine which magnetic field sensing assembly of the plurality of magnetic field sensing assemblies senses the strongest magnetic field from the permanent magnet.

Example 63. The system of Example 61, wherein each magnetic field sensing assembly of the plurality of magnetic field sensing assemblies is configured to be positioned at a known position relative to the subject.

Example 64. The system of any of Examples 55-58, further comprising: a display device configured to display a graphical representation of the pose of the instrument based on the magnetic field sensed by the magnetic field sensing assembly of the permanent magnet.

Example 65. A method to determine a pose of an instrument, comprising: providing a permanent magnet to generate a magnetic field that defines a magnet axis; providing a magnetic field sensing assembly comprising, a first magnetic field sensor configured to sense the magnetic field relative to a first axis relative to the magnetic field sensing assembly, a second magnetic field sensor configured to sense the magnetic field relative to a second axis relative to the magnetic field sensing assembly, and a third magnetic field sensor configured to sense the magnetic field relative to a third axis relative to the magnetic field sensing assembly, positioning the first axis, the second axis, and the third axis orthogonal to each other within the magnetic field sensing assembly; and executing instructions with a processing unit to: receive a signal from the magnetic field sensor assembly regarding the sensed magnetic field relative to each of the first axis, the second axis, and the third axis, and determine the pose of the instrument.

Example 66. The method of Example 65, further comprising: providing the instrument including the permanent magnet incorporated therewith; sizing the instrument to be inserted within at least a portion of a subject.

Example 67. The method of Example 66, wherein sizing the instrument to be inserted within at least the portion of the subject includes sizing the instrument to be inserted within a vessel of a human subject.

Example 68. The method of Example 65, wherein providing the permanent magnet includes providing a plurality of permanent magnets; and positioning each permanent magnet of the plurality of permanent magnets spaced apart from the other permanent magnet(s) of the plurality along the instrument.

Example 69. The method of Example 68, wherein providing the permanent magnet includes providing a single permanent magnet having the magnetic field that is sensed to determine the pose of a selected portion of the instrument.

Example 70. The method of Example 65, wherein providing the magnetic field sensing assembly includes providing a plurality of magnetic field sensing assemblies.

Example 71. The method of Example 70, wherein executing the instructions with the processing unit includes executing further instructions to determine which magnetic field sensing assembly of the plurality of magnetic field sensing assemblies senses the strongest magnetic field from the permanent magnet.

Example 72. The method of Example 71, wherein executing instructions with the processing unit to determine the pose of the instrument includes determining the pose of the instrument based on the signal from the magnetic field sensing assembly determined to sense the strongest magnetic field.

Example 73. The method of Example 70, further comprising: determining a position relative to a subject of each of the magnetic field sensing assemblies of the plurality of magnetic field sensing assemblies.

Example 74. The method of Example 65, further comprising: displaying with a display device a graphical representation of the pose of the instrument based on the magnetic field sensed by the magnetic field sensing assembly of the permanent magnet.

What is claimed is:

1. A system to assist a user in navigating an instrument relative to a subject, comprising:
a display device configured to be viewed by the user;
an instrument tracking device associated with the instrument;
a subject tracking device, wherein the subject tracking device is fixed relative to the subject;
a tracking system configured to track the instrument tracking device and the subject tracking device; and
a processor configured to execute instructions to:
determine a pose of the instrument relative to a pose of the subject based on the tracking of the instrument tracking device and the subject tracking device;
receive information regarding the subject;
recall a standard avatar;
generate a subject specific avatar at least by morphing the recalled standard avatar based on the received information regarding the subject; and
generate output that causes the display device to display the subject specific avatar and a graphical representation of the instrument superimposed on the subject specific avatar based on the pose of the instrument relative to the pose of the subject.

2. The system of claim 1, further comprising the instrument, wherein the instrument comprises a medical device.

3. The system of claim 2, wherein the instrument comprises an elongate endovascular device.

4. The system of claim 2, wherein the instrument comprises an intracardiac device.

5. The system of claim 2, wherein the instrument comprises a guidewire.

6. The system of claim 2, wherein the instrument comprises a catheter.

7. The system of claim 2, wherein the instrument comprises a delivery system for an implant.

8. The system of claim 2, wherein the instrument comprises a tunneling tool.

9. The system of claim 2, wherein the instrument comprises an energy delivery device.

10. The system of claim 2, wherein the instrument comprises an electrode configured to stimulate a heart of the subject.

11. The system of claim 1, wherein the processor is configured to execute further instructions to maintain a registration of the subject specific avatar with the subject based on the tracking of the subject tracking device.

12. The system of claim 1, wherein the subject tracking device is configured to be positioned at the suprasternal notch of the subject.

13. The system of claim 1, wherein the processer is configured to determine poses of at least two points of the subject.

14. The system of claim 1, wherein the tracking system includes a localizer, wherein the subject is positioned relative to the localizer at a predetermined position, and wherein the predetermined position is operable to allow registration of the subject to the subject specific avatar.

15. A method to assist a user in navigating an instrument relative to a subject by selecting and sizing an avatar of a subject, the method comprising:
tracking, by a tracking system, an instrument tracking device associated with the instrument and a subject tracking device that is fixed relative to the subject;
determining, by a processor, a pose of the instrument relative to a pose of the subject based on the tracking of the instrument tracking device and the subject tracking device;
receiving, by the processer, information regarding the subject;
recalling, by the processor, a standard avatar;
generating, by the processor, a subject specific avatar at least by morphing the standard avatar based on the received information regarding the subject; and
generating, by the processing, output that causes a display device to display the subject specific avatar and a graphical representation of the instrument superimposed on the subject specific avatar based on the pose of the instrument relative to the pose of the subject.

16. The method of claim 15, wherein generating the subject specific avatar comprises generating the subject specific avatar to have subject portions based on relative positions of the subject portions in the subject.

17. The method of claim 15, further comprising maintaining, by the processor, a registration of the subject specific avatar with the subject based on the tracking of the subject tracking device.

18. The method of claim 15, wherein the subject tracking device is positioned at the suprasternal notch of the subject.

19. The method of claim 15, wherein determining the pose of the subject comprises determining poses of at least two points of the subject.

20. The method of claim 15, wherein the tracking system comprises a localizer positioned at a predetermined position relative to the subject, the method comprising registering, by the processor, the subject to the subject specific avatar based on the predetermined position relative to the subject of the localizer.
